(19) United States
(12) Patent Application Publication
(10) Pub. No.: US 2025/0034496 A1
(43) Pub. Date: Jan. 30, 2025

(54) Pancreatic Cancer Organoids-on-a-Chip

(71) Applicant: NEW YORK UNIVERSITY, New York, NY (US)

(72) Inventors: Weiqiang Chen, New York, NY (US); Lunan Liu, Brooklyn, NY (US)

(21) Appl. No.: 18/785,711

(22) Filed: Jul. 26, 2024

Related U.S. Application Data
(60) Provisional application No. 63/516,291, filed on Jul. 28, 2023.

(51) Int. Cl. C12M 3/00; C12M 3/06; G01N 33/50

(52) U.S. Cl. CPC C12M 21/08; C12M 23/16; G01N 33/5011

(57) ABSTRACT

Aspects of the present invention relate to a pancreatic cancer organoids on a chip device including a microfluidic chip having a top and bottom surface and a thickness therebetween, a central chamber embedded in the microfluidic chip, at least one opening in the top surface of the chip fluidly connected to the central chamber with one or more channels, and a plurality of evenly spaced micropillars arranged in a substantially circular shape within the central chamber such that the central chamber is partitioned into at least a first inner region and a first outer region, wherein the first inner region includes an immunocompetent pancreatic region configured to mimic a pancreatic niche, and the first outer region includes a vascular region configured to mimic an adjacent vascular network.

### CROSS-REFERENCE TO RELATED APPLICATIONS

This application claims priority to U.S. Provisional Application No. 63/516,291 filed on Jul. 28, 2023, incorporated herein by reference in its entirety.

### BACKGROUND OF THE INVENTION

Pancreatic ductal adenocarcinoma (PDAC) is the most common type of pancreatic cancer and is highly malignant with an overall 5-year survival rate of about 10%. PDAC is projected to be the 2^{nd} leading cause of cancer related deaths by the year 2030. Current treatments like surgery, chemotherapy, and immune checkpoint inhibitors are only suitable for a small subset of patient with low response rates. Thus, there remains an urgent and unmet need to develop safe and effective therapies for pancreatic disease and PDAC patient treatment.

However, despite numerous immunotherapies having shown remarkable success in other cancers—pancreatic cancer seemed to be intrinsically resistant to immunotherapy, which is largely attributed to its “immune cold” tumor microenvironment (TME). The tumor immunosuppression and microenvironmental heterogeneity are emerging as potential barriers in efficient PDAC treatment. The PDAC TME is a “milieu” of distinct elements including altered extracellular matrix (ECM), fibroinflammatory stroma (cancer-associated fibroblasts (CAFs)), infiltrating immunosuppressive cells (i.e., tumor-associated macrophages (TAMs), regulatory T (Treg) cells, and myeloid-derived suppressor cells (MDSCs)), and includes an aberrant expression of checkpoint pathway proteins, immunosuppressive cytokines and molecules. The desmoplasia in PDAC TME modeled by activated CAFs results in dense stroma, lack of vasculature, hypoxia and high interstitial flow pressure (IFP) in PDAC, aggravating TME immunosuppression and possibly thwarting T-cell extravasation and permitting tumor escape from immune surveillance.

Additionally, PDAC is a genetically heterogeneous disease and can be classified as a poorly differentiated aggressive basal-like subtype, and a classical subtype with a better prognosis. Basal-like subtype demonstrates an “immune escape” mechanism with a more hostile TME richer in M2-polarized TAMs and Treg cells and poor in effector T cells. However, classical subtype displays an “immune rich” and cytotoxic TME, characterized with more effector T cells and M1-polarized TAMs, and a lower level of Treg cells and M2-polarized TAMs. The subtype-specific immune niche further results in the heterogeneous therapeutic responses in PDAC. Therefore, current treatments are only suitable to a small subset of patients with low response rates.

Thus, there is a need in the art for an accurate, real-time, and modular device and method to reproduce and understand the highly heterogeneous and immunosuppressive TME of PDAC to help test and screen promising therapies mimicking the clinical situation. The present invention meets this need.

### SUMMARY OF THE INVENTION

Aspects of the present invention relate to a pancreatic cancer organoids on a chip device including a microfluidic chip having a top and bottom surface, a central chamber embedded in the microfluidic chip, at least one opening in the top surface of the chip fluidly connected to the central chamber with one or more channels, and a plurality of evenly spaced micropillars arranged in a substantially circular shape within the central chamber such that the central chamber is partitioned into at least a first inner region and a first outer region, wherein the first inner region includes an immunocompetent pancreatic region configured to mimic a pancreatic niche, and the first outer region includes a vascular region configured to mimic an adjacent vascular network.

In some embodiments, the immunocompetent pancreatic region includes a 3D pancreatic tumor niche. In some embodiments, the first outer region further includes a vascular region configured to serve as a pathway for nutrients, drugs and cells transport.

In some embodiments, the first inner region includes one or more components selected from the group consisting of: hydrogel, pancreatic ductal adenocarcinoma (PDAC) cells, patient-derived organoids (PDOs), primary human umbilical vein endothelial cells (HUVECs), green fluorescent protein (GFP) expressing human umbilical vein endothelial cells (GFP-HUVECs), vasculature, extracellular matrix (ECM), culture media, cytokines, cancer-associated fibroblasts (CAFs), chimeric antigen receptor (CAR) T-cells, immune cells (tumor associated macrophages (TAMs), Treg cells, and myeloid-derived suppressor cells (MDSCs)).

In some embodiments, the first outer region includes components selected from the group consisting of: primary human umbilical vein endothelial cells (HUVECs), green fluorescent protein (GFP) expressing human umbilical vein endothelial cells (GFP-HUVECs), vascular niche supporting cells: normal human lung fibroblasts (NHLFs), hydrogel, endothelial cells growth medium (EGM-2, Lonza), and Vascular endothelial growth factor (VEGF).

In some embodiments, the device is configured to replicate or mimic a pancreatic disease or disorder state selected from the group consisting of: pancreatic disease, pancreatitis, pancreatic cancer, and pancreatic ductal adenocarcinoma (PDAC). In some embodiments, the device includes CAR T-cells in the first outer region.

In some embodiments, the first inner region and the first outer region are concentric. In some embodiments, the plurality of micropillars are evenly spaced by a distance between about 50 μm and 200 μm and have a cross-sectional shape selected from the group consisting of: circular, ovoid, square, rectangular, triangular, trapezoidal, and polygonal.

Aspects of the present invention relate to a method of determining pancreatic disease treatment responsiveness, having the steps of providing any microfluidic device of the present invention, administering a pancreatic disease treatment to the central chamber, and determining pancreatic disease treatment responsiveness based on a measured change in the central chamber.

In some embodiments, the pancreatic disease treatment is a therapy selected from the group consisting of: anti-cancer drug, immunotherapy, chemotherapy, radiation therapy, chemoradiation therapy, and targeted therapy.

In some embodiments, the measured change comprises measuring a change in one or more parameters selected from the group consisting of: CAR T-cell infiltration frequency, speed, trajectory, and distance, expression levels of activation (CD25, CD154, CD69), proliferation (Ki67), cytotoxic function (interferon gamma, granzyme B and perforin) markers, relative frequencies and immunosuppressive function of TAM (CD163, Fizz-1, Arg1, CSF-1) and Treg cell (CD4⁺CD25⁺Foxp3⁺, CTLA-4), immunosuppressive cytokine profile (e.g., TGF-β, IL-10, CCL2), expression level of immune checkpoints markers (e.g. PD1/PDL1), the expression level of targeted antigens on tumor and the apoptosis rate of tumor cells.

In some embodiments, the first inner region and the first outer region are concentric. In some embodiments, the plurality of micropillars are evenly spaced by a distance between about 50 μm and 200 μm and have a cross-sectional shape selected from the group consisting of: circular, ovoid, square, rectangular, triangular, trapezoidal, and polygonal.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1 depicts an “Immune cold” pancreatic ductal adenocarcinoma (PDAC) tumor microenvironment that may hinder immunotherapy (e.g., chimeric antigen receptor (CAR) T-cell therapy) outcome.

FIGS. 2A & 2B depict an exemplary patient-derived ‘PDAC Organoids-on-a-Chip’ microfluidic device and model for immunotherapy (e.g., CAR T-cell therapy) screening and optimization according to aspects of the present invention.

FIGS. 3A through 3C show aspects of an exemplary PDAC organoids-on-a-chip device comprising PDAC organoids and vasculature on chip. FIG. 3A shows a 3D and 2D local view of the PDAC organoids. FIG. 3B shows a 3D view and infused dextran showing the intact and perfusable vasculature on chip. FIG. 3C shows a whole scan and the enlarged views of the PDAC organoids-on-a-chip.

FIG. 4 shows immunostaining results showing the upregulation of markers for different types of cancer-associated fibroblasts (CAFs): myCAFs (α-SMA), iCAFs (PDGFRα), apCAFs (CD74) after Patient Derived Organoids (PDOs) were loaded on the microfluidic chip device according to aspects of the present invention.

FIG. 5 shows results indicating that activated PSCs induce dense ECM with excessive collagen I (COL I).

FIGS. 6A through 6D show the microfluidic chip device generated hypovascularized stromal niche modeled by PDAC. FIG. 6A shows the characterizations of vascularity and FIG. 6B shows dynamic changes in height and vessel area of the vasculature. FIG. 6C shows the intactness of the vessel accessed by quantifying the junctions formed by HUVECs on vascular walls (staining CD31) and FIG. 6D shows apoptosis of vasculature induced by PDOs.

FIGS. 7A through 7D depict exemplary microfluidic microenvironmental regulator units for hypoxia, fluids, and interstitial fluid pressure control, and an exemplary gas mixer according to aspects of the present invention. FIG. 7A depicts an exemplary microfluidic chip device with microenvironmental regulator unit comprising a gas layer (green), a pressure regulation layer (pink), a valve control layer (red) and a culture layer (blue). FIG. 7B depicts an exemplary pressure regulator unit (Scale bar: 50 μm). FIG. 7C depicts an exemplary device layout (e.g., a plurality of channels) comprising an oxygenator layer (green), a pressure regulation layer (blue), a valve control layer (red) and a culture layer (gray). FIG. 7D depicts an exemplary gas mixer which regulates the oxygen concentration by defining fluidic resistances of two inlet channels (R1 and R2).

FIG. 8 depicts an exemplary organotypic immune niche model recapitulating the immunosuppressive PDAC tumor microenvironment (TME).

FIGS. 9A through 9H show the microfluidic chip device of the present invention can characterize the immunosuppressive PDAC TME. FIG. 9A shows immune niche comprising PDOs and immunosuppressive TAMs (CD68⁺) and Treg cells (FOXP3⁺). FIG. 9B shows the upregulation of CD163 (marker for M2-like macrophages), FIG. 9C shows the ratio of CD163 to HLA-DR (marker for M1-like macrophages), and FIG. 9D shows the ratio of M2 to M1-like macrophage as PDOs exist, indicating the immunosuppression induced by PDAC on chip. FIG. 9E shows the results for examining the subtype-specific biomarkers CK17 (basal-like) and GATA6 (classical) on two PDOs. FIGS. 9F, 9G, and 9H show the results comparing immune checkpoints PD-1, CTLA-4, and PD-L1 in immune niches of two different PDOs on chip.

FIGS. 10A through 10C show that the microfluidic chip device of the present invention investigated CAFs-mediated formation of immunosuppressive PDAC TME. FIG. 10A depicts measurements of continuous upregulation of IL-10 after PDOs were loaded especially with PSC on chip. FIG. 10B shows that TGF-β increases as PDOs exist. FIG. 10C shows that upregulations of tumor and PSC induced cytokines indicate immunosuppressive TME.

FIGS. 11A through 11F show the microfluidic chip device of the present invention investigated the function of CAR T-cells and induced immune responses. FIGS. 11A through 11B show that CAR T-cells showed a higher level of activation marker (FIG. 11A) and proliferation marker (FIG. 11B) comparing with those of MOCK T-cells and demonstrated increasements from day 3 to day 6. FIGS. 11C through 11D show that through the on-chip vascular network, CAR T-cells showed extravasation (FIG. 11C), infiltration and killing abilities (FIG. 11D). FIG. 11E shows that higher infiltration of CAR T-cells towards PDOs were observed from day 3 to day 6 while MOCK T-cells had no improvement. FIG. 11F shows that CAR T-cells induced the highest level of inflammatory responses compared with no T-cells or with MOCK T-cells.

FIGS. 12A through 12G show the microfluidic chip device of the present invention investigated patient-specific CAR T-cell therapy response. FIGS. 12A through 12D show live/dead cell staining results (FIG. 12A) showing patient-specific CAR T-cell therapy response (FIGS. 12B & 12C) and its correlation with the expression level of targeted antigen MSLN (FIG. 12D). FIGS. 12E through 12G show immunofluorescence results (FIG. 12E) indicating the different expression levels of various tumor antigens between two patients (FIGS. 12F & 12G).

### DETAILED DESCRIPTION

The present invention relates to devices that replicate pancreatic tissue and/or pancreatic tumor microenvironment (TME) in a microfluidic chip, and associated methods of use. The devices can be used to model certain disease states related to the pancreas, such as pancreatic ductal adenocarcinoma (PDAC). The devices can be adapted to replicate the microenvironment from patient-specific cells such that treatment conditions can be modeled and tailored to individual patients. In some embodiments, the devices are suitable for evaluating any therapy including, but not limited to, anti-cancer drug, immunotherapy, chemotherapy, radiation therapy, chemoradiation therapy, and targeted therapy on a patient-specific basis.

It is to be understood that the figures and descriptions of the present invention have been simplified to illustrate elements that are relevant for a clear understanding of the present invention, while eliminating, for the purpose of clarity many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Pancreatic Cancer Organoids-on-a-Chip

Disclosed in some examples is a patient-derived “Pancreatic ductal adenocarcinoma (PDAC) Organoids-on-a-Chip” micro-physiological device formed by strategically integrating the state-of-the-art organoids and organ-on-a-chip technologies to accurately reconstitute the pathophysiology and heterogeneity of the PDAC tumor microenvironment (TME) in vitro to study mechanisms of resistance; and assess, optimize and personalize different therapies. The disclosed device integrates two emerging technologies, i.e., PDAC patient derived organoids (PDOs) and a vascularized TME chip with biomimetic stromal and immune niches. The device addresses the limitations of each individual approach, and synergistically combines the best features of each approach to provide a more powerful in vitro technology for PDAC therapy modeling and screening. In some embodiments, the disclosed device utilizes patient-derived tumor organoids (PDOs) that can maintain molecular characteristics of the original tumor and allows for assessing new anticancer drugs, while also preserving the context of the patient's TME immune signatures and architectures (i.e., tumor vasculature, immune and stromal cells) that confer therapeutic resistance. Although exemplary microfluidic chip devices are shown and described herein, it should be understood that the present invention may be utilized on or with any microfluidic chip, organoid-on-chip, and micro-physiological chip that would be known by one of ordinary level of skill in the art—and the terms may be used interchangeably herein.

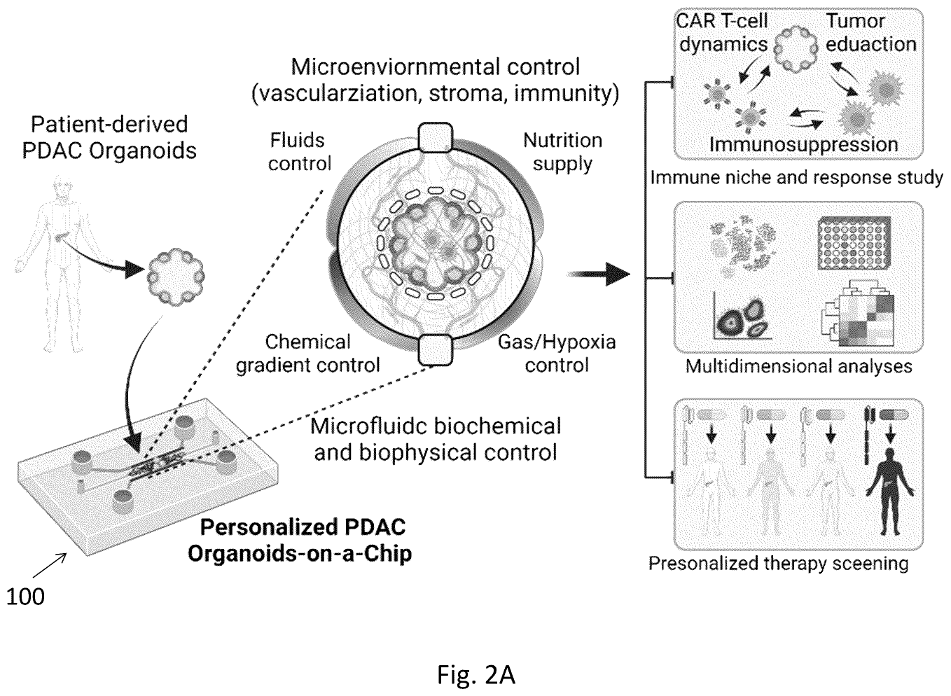

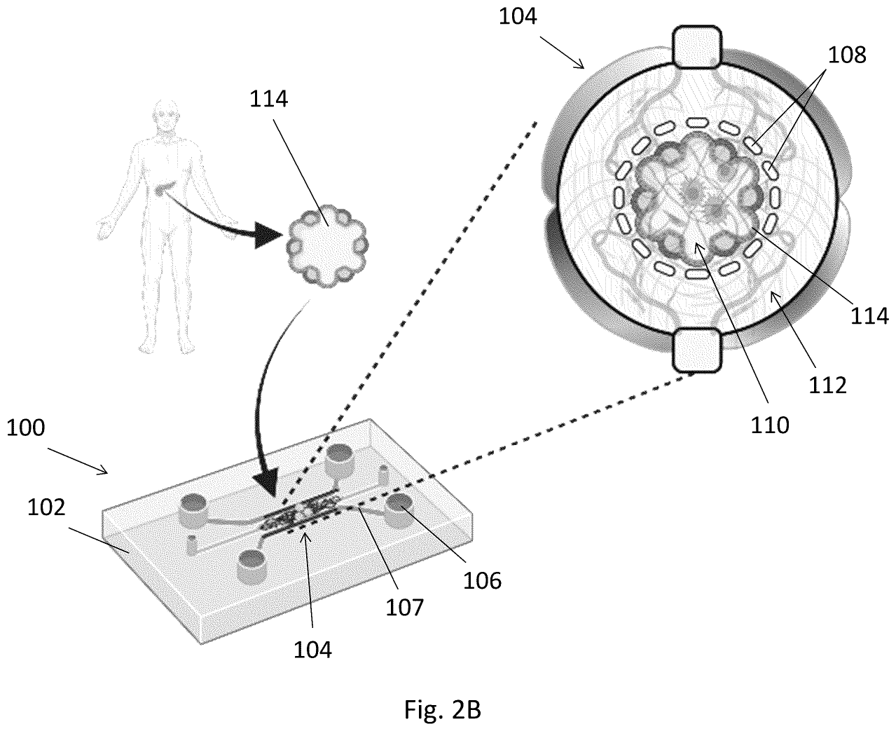

Aspects of the present invention relate to an organoids-on-a-chip micro-physiological device, in some examples referred to interchangeably as a pancreas-on-a-chip device, a pancreas organoids-on-a-chip device, a pancreatic cancer organoids-on-chip device, or a PDAC organoids-on-a-chip device. Now referring to FIG. 2B, shown is an exemplary pancreas organoids-on-a-chip device 100 comprising a microfluidic chip 102 comprising a top and bottom surface and a thickness or height, a central chamber 104 embedded in the top surface of microfluidic chip 102, one or more openings 106 in the top surface of the chip fluidly connected to central chamber 104 with one or more channels 107. Now referring to the enlarged view in FIG. 2B, in some embodiments, microfluidic chip 102 further comprises a plurality of micropillars 108 arranged within central chamber 104 such that central chamber 104 is partitioned into at least a first inner region 110 and a first outer region 112, in some examples referred to as one or more culture compartments. In some embodiments, plurality of micropillars 108 are evenly spaced in central chamber 104 and arranged in a substantially circular shape. In some embodiments, first inner region 110 and first outer region 112 are concentric. In some embodiments, the plurality of micropillars 108 are spaced by a distance between about 50 μm and 200 μm and have a shape (e.g., a cross-sectional shape) selected from the group consisting of: circular, ovoid, square, rectangular, triangular, trapezoidal, and polygonal.

Aspects of the present invention relate to device 100 comprising biomimetic components and/or features. In some embodiments, first inner region 110 comprises an immunocompetent pancreatic region configured to mimic a pancreatic niche. In some embodiments, first outer region 112 comprises a vascular region configured to mimic an adjacent vascular network. In some embodiments, the immunocompetent pancreatic region comprises a 3D pancreatic tumor niche. In some embodiments, first inner region 110 comprises one or more patient-derived organoids (PDOs) 114. In some embodiments, first inner region 110 further comprises a vascular region configured to serve as a pathway for nutrients, drugs and cells transport.

Aspects of the present invention relate to device 100 comprising one or more cells and/or components in central chamber 104, first inner region 110 and/or first outer region 112. In some embodiments, first inner region 110 comprises one or more components selected from the group consisting of: hydrogel, pancreatic ductal adenocarcinoma (PDAC) cells, patient-derived organoids (PDOs), primary human umbilical vein endothelial cells (HUVECs), green fluorescent protein (GFP) expressing human umbilical vein endothelial cells (GFP-HUVECs), extracellular matrix (ECM), culture media, cytokines, cancer-associated fibroblasts (CAFs), chimeric antigen receptor (CAR) T-cells, immune cells (tumor associated macrophages (TAMs), Treg cells, and myeloid-derived suppressor cells (MDSCs)). In some embodiments, the PDOs are sourced from the human Cancer Models Initiative of the American Type Culture Collection (ATCC), National Cancer Institute (NCI) Patient-Derived Models Repository (PDMR), and NYU Langone Health. In some embodiments, the PDOs are sourced from any institutional cancer center. In some embodiments, the PDOs are sampled from a subject. In some embodiments, the PDOs are sampled from a biopsy. In some embodiments, the PDOs are samples from resections or needle biopsies. In some embodiments, the PDOs sampled from resections or needle biopsies are minced into small fragments and then digested into small cell clusters (less than 10 cells) or single cells. In some embodiments, the digested small cell clusters or single cells are embedded in basement membrane (e.g., Matrigel, ATCC cell basement membrane). In some embodiments, the digested small cell clusters or single cells are cultured in organoids culture mediums to grow into organoids. In some embodiments, the PDOs are from Human Cancer Models Initiative of ATCC, National Cancer Institute (NCI) Patient-Derived Models Repository (PDMR), NYU Langone Health, or from any institutional cancer center.

In some embodiments, first outer region 112 comprises components selected from the group consisting of: primary human umbilical vein endothelial cells (HUVECs), green fluorescent protein (GFP) expressing human umbilical vein endothelial cells (GFP-HUVECs), vascular niche supporting cells: normal human lung fibroblasts (NHLFs), hydrogel, endothelial cells growth medium (EGM-2, Lonza), and Vascular endothelial growth factor (VEGF). In some embodiments, device 100 comprises CAR T-cells in first outer region 112.

Aspects of the present invention relate to replicating the conditions of a specific disease state and/or tumor microenvironment within device 100. In some embodiments, device 100 is configured to replicate or mimic a pancreatic disease or disorder state selected from the group consisting of: pancreatic disease, pancreatitis, pancreatic cancer, cystic fibrosis, and pancreatic ductal adenocarcinoma (PDAC). In some embodiments, device 100 is configured to provide a hypoxic environment and high interstitial fluid pressure (IFP) to the pancreatic niche.

In some embodiments, device 100 comprises and/or is configured with an in vitro tumor model. In some embodiments, device 100 is tissue engineered with a central vascularized immunocompetent PDAC niche (e.g. in first inner region 110) comprising PDAC patient-derived organoids (PDOs), 3D vascular network, cancer-associated fibroblasts (CAFs), niche immune cells (tumor-associated macrophages (TAMs), Treg cells, and myeloid-derived suppressor cells (MDSCs)), and an adjacent vascular region (e.g. in first outer region 112) to serve as the interface of tumor-stroma-immune interactions. In some embodiments, device 100 comprises one or more culture compartments (e.g. first inner region 110, and first outer region 112) that are segregated by regularly spaced polydimethylsiloxane (PDMS) micropillars 108 that restrict cell-embedded hydrogel to simulate the in vivo architecture of the TME. In some embodiments, device 100 comprises media reservoirs for long-term media supply (e.g. in openings 106).

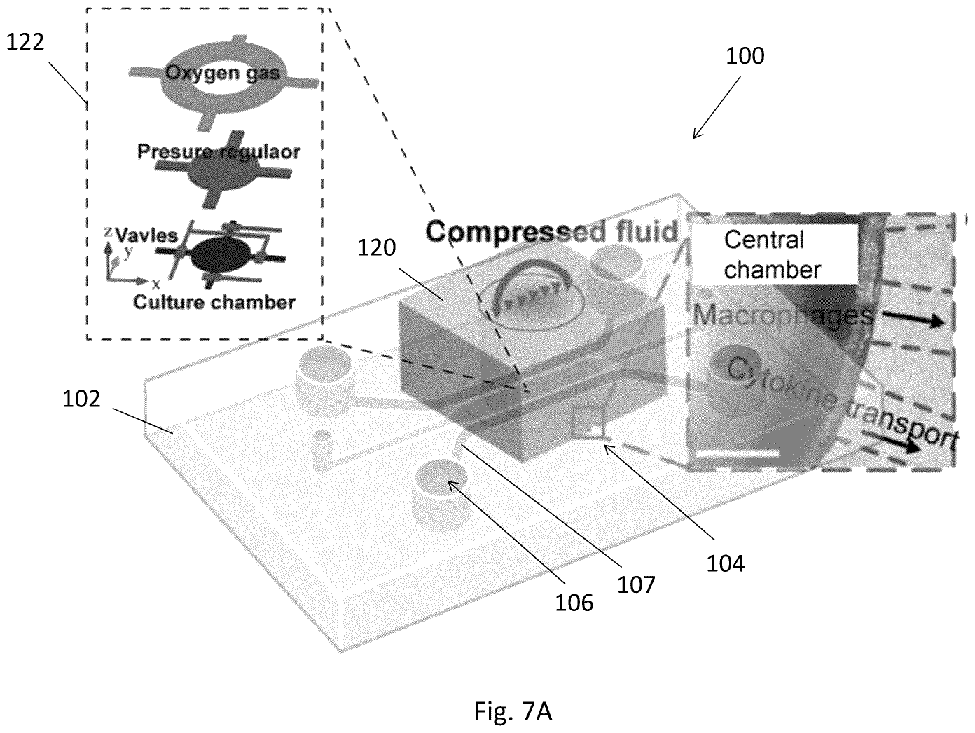

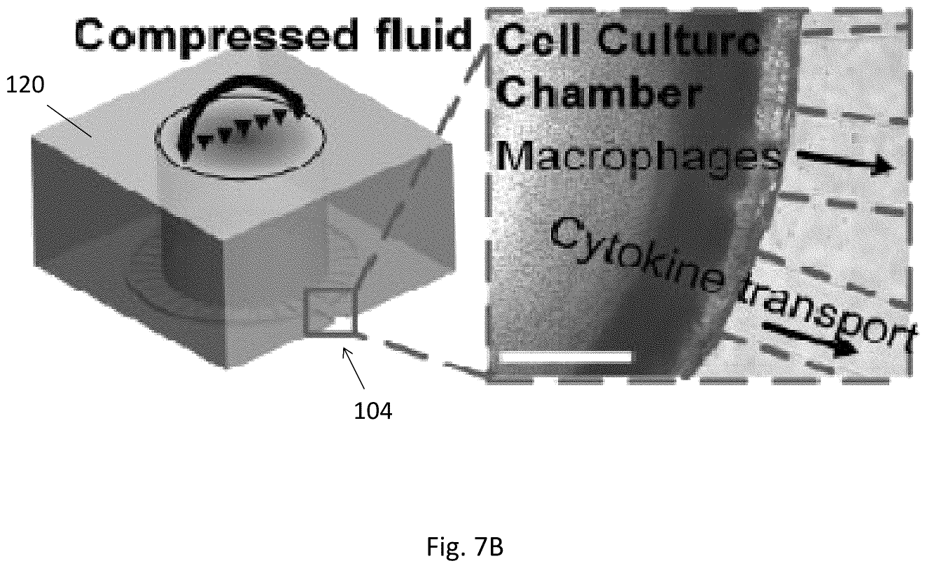

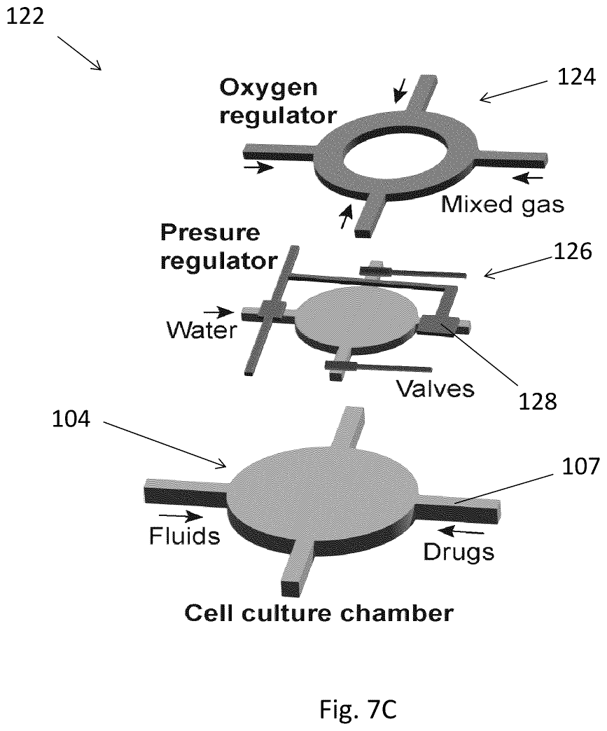

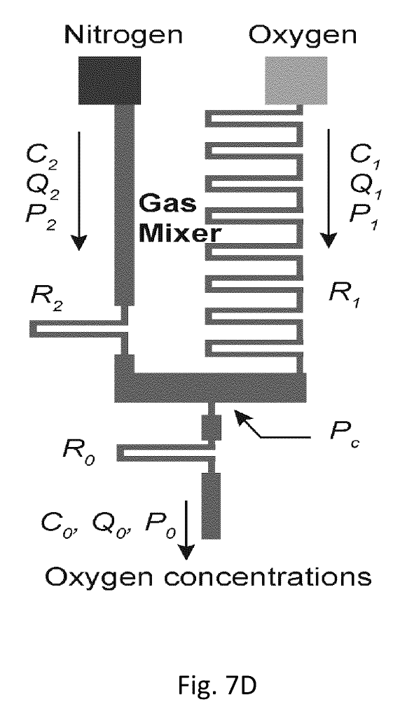

Aspects of the present invention relate to a microfluidic microenvironmental regulator unit for an organoid-on-chip device (e.g., device 100). In some embodiments, device 100 comprises one or more microfluidic microenvironmental regulators connected to microfluidic chip 102. In some embodiments, the one or more microfluidic microenvironmental regulators are integrated surrounding and/or on top of tissue culture chamber (e.g. central chamber 104). Referring now to FIGS. 7A through 7C, shown is an exemplary microfluidic microenvironmental regulator 120 configured to fixedly and sealingly attach to the top surface of microfluidic chip 102 and at least partially enclose central chamber 104. In some embodiments, microfluidic microenvironmental regulator unit 120 creates and/or controls hypoxic conditions for device 100 and/or central chamber 104. In some embodiments, microenvironmental regulator unit 120 provides control for fluids provided to device 100 and/or central chamber 104 with a plurality of channels 122. In some embodiments, microenvironmental regulator unit 120 provides control for and/or is configured to provide interstitial fluid pressure (IFP). In some embodiments, plurality of channels 122 comprises an oxygen gas channel 124, a pressure regulator channel 126, and a plurality of valves 128 fluidly connected to openings 106, channels 107 and/or central chamber 104 or device 100. In some embodiments, microenvironmental regulator unit 120 and/or plurality of channels 122 sits over top of device 100 and/or central chamber 104 and sealingly close the central opening of the device while leaving the peripheral openings (e.g., openings 106) on chip 102 open.

In some embodiments, device 100 comprises key stroma and immune niche factors in central chamber 104 (in some examples referred to as the PDAC TME) with controllable biological features (e.g. cell type and density, extra-cellular matrix (ECM) components, biophysical parameters like oxygen level and IFP). In some embodiments, device 100 recapitulates a TME with patient-specific organoids and/or PDOs and patient-specific niche cells (e.g., CAFs, TAMs, Tregs and MDSCs). In some embodiments, device 100 maintains the original patient-specific features in PDOs and niche cells with high pathological relevance. In some embodiments, device 100 enables real-time characterization of pathophysiological processes (e.g. cancer cells apoptosis, cytotoxic cells proliferation, activation and trafficking) at high spatiotemporal resolution.

In some embodiments, device 100 comprises one or more third-party biological materials and/or PDOs positioned within central chamber 104. In some embodiments, the one or more third-party biological materials and/or PDOs comprise PDAC PDOs line PDM-40, PDM-41, sourced from Human Cancer Models Initiative of ATCC, PDO lines 242566, 485368, 671287, 868679, 982776 from NCI PDMR, and/or PDOs lines NYU318, NYU341, NYU421, NYU429, NYU455 from NYU Langone Health, or PDOs from any institutional cancer center. In some embodiments, the PDOs are cultured in Cell Basement Membrane (ATCC) or Matrigel (Corning) or Cultrex Reduced Growth Factor Basement Membrane Extract, Type R1 (R&D Systems). In some embodiments, device 100 comprises one or more organoid culture mediums comprising one or more components positioned within central chamber 104. In some embodiments, the organoid culture medium comprises PancreaCult Organoid Media (Human) (STEMCELL) or any known culture medium. In some embodiments, the components of the one or more organoid culture mediums comprise any of Advanced DMEM:F12 (Thermo Fisher), HEPES (Thermo Fisher), B-27 supplement (Thermo Fisher), GlutaMAX™ (Thermo Fisher) or L-Glutamine (ATCC), noggin (Bio-techne), EGF (Bio-techne), Gastrin (Bio-techne), FGF-10 (Bio-techne), A83-01 (Bio-techne), nicotinamide (Sigma or LKT Labs), N-acetyl cysteine (Sigma or LKT Labs), RSPO-1 conditioned medium (Sigma)/Recombinant Human R-Spondin 1 Protein (R&D Systems) and Wnt-3A conditioned medium (MBL)/Recombinant Afamin/Wnt3a (MBL) and Y27632 (ATCC or Abmole).

In some embodiments, device 100 comprises one or more cells cultured in or on the one or more organoid culture mediums. In some embodiments, the one or more cells comprise Pancreatic Stellate Cells (PSCs, a major source of CAFs, ScienCell) cultured in Stellate Cell Medium (SteCM, ScienCell) or patient-derived CAFs cultured in RPMI 1640 medium (Thermo Fisher), primary human umbilical vein endothelial cells (HUVECs, Lonza) or primary GFP expressing human umbilical vein endothelial cells (GFP-HUVECs, Angioproteomie) cultured in endothelial cells growth medium (EGM-2, Lonza) and/or vascular niche supporting cells (normal human lung fibroblasts, NHLFs, Lonza) cultured in fibroblast cells growth medium (FGM-2, Lonza) mixed in a fibrinogen hydrogel (Sigma).

In some embodiments, the one or more cells comprise monocytes isolated from healthy donor-derived peripheral blood mononuclear cells (PBMCs, iXCells Biotechnologies), or patient-derived PBMCs using Human Pan Monocyte Isolation Kit (Biolegend) and treated with 25 ng/mL MCSF (R&D Systems) for 7 days to mimic CD14⁺CD68⁺ TAMs. In some embodiments, the one or more cells comprise CD4⁺ T cells isolated using Human Naive CD4⁺ T Cell Isolation Kit (R&D Systems) and differentiated into CD4⁺ CD25⁺ FOXP3⁺ Treg cells by Human Treg Cell Differentiation Kit (R&D Systems). In some embodiments, the one or more cells comprise CD11b⁺CD14⁻CD15⁺ MDSCs isolated using the Cell Isolation Kits (Miltenyi Biotec).

### Fabrication

Aspects of the present invention relate to the fabrication of an exemplary organoids-on-a chip device (e.g., device 100). In some embodiments, device 100 is fabricated using standard PDMS soft lithography technique [Cui X et al., Dissecting the immunosuppressive tumor microenvironments in Glioblastoma-on-a-Chip for optimized PD-1 immunotherapy. Elife. 2020; 9:e52253; Ma C et al., Leukemia-on-a-chip: Dissecting the chemoresistance mechanisms in B cell acute lymphoblastic leukemia bone marrow niche. Sci Adv. 2020; 6(44)]. It should be appreciated herein that device 100 and/or microfluid chip 102 may be formed from any suitable material known for microfluidic chips by one of ordinary level of skill in the art, and microfluid chip 102 may have any suitable size, shape and/or dimensions. In some embodiments, to populate or form device 100, PDAC organoids and different niche cells are compartmentalized on the device in different culture compartments (i.e. first inner region 110 and first outer region 112) following a multiple-step loading and self-assembly protocol [Ma C et al., Leukemia-on-a-chip: Dissecting the chemoresistance mechanisms in B cell acute lymphoblastic leukemia bone marrow niche. Sci Adv. 2020; 6(44)].

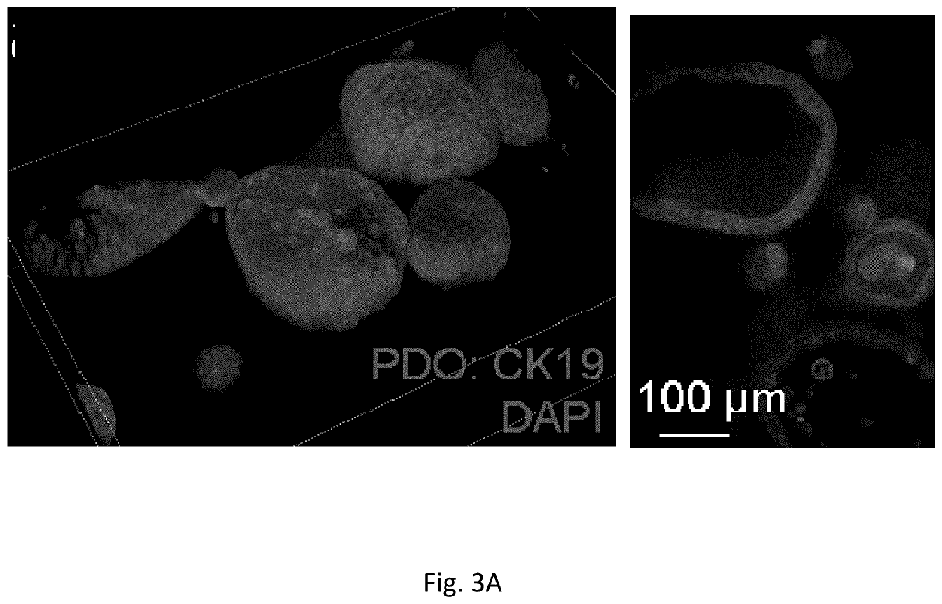

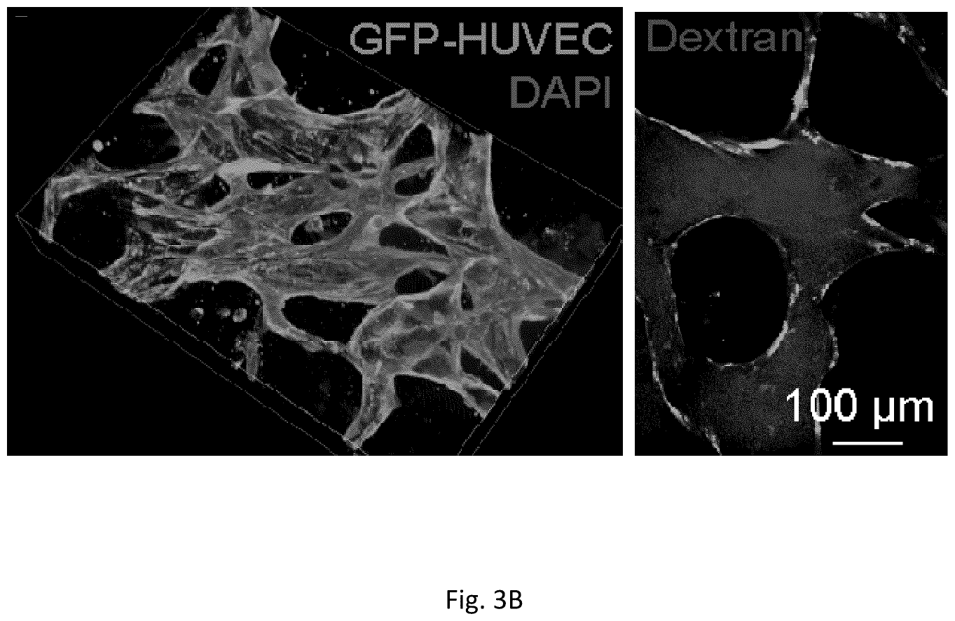

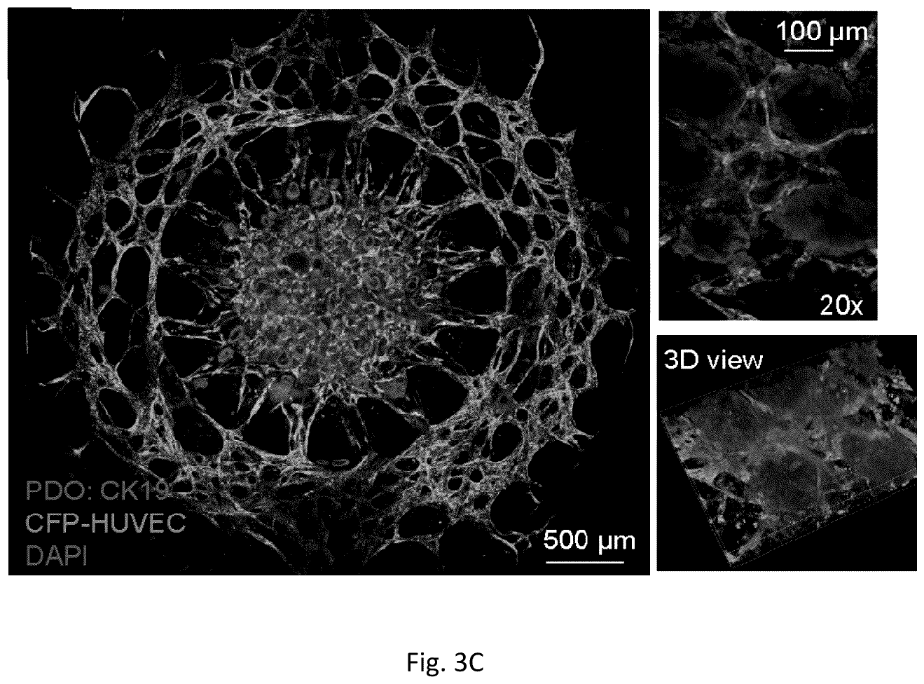

In some embodiments, device 100 recapitulates a vascularized stromal niche on chip (e.g., in central chamber 104). In some embodiments, to recapitulate the vascularized stromal niche, primary GFP expressing human umbilical vein endothelial cells (GFP-HUVECs, Angioproteomic) and vascular niche supporting cells (normal human lung fibroblasts, NHLFs, Lonza) with optimized seeding density for each cell type (e.g. 10⁶-10⁷ cells/ml) are loaded in a fibrinogen hydrogel (3 mg/mL, Sigma) in the outer ring (first outer region 112) of the device 100 (FIGS. 3C & 6A). In some embodiments, EGM-2 (Lonza) medium with 50 ng/mL VEGF has been validated to support the growth of stromal cells on device 100. Based on the disclosed configuration and results (FIGS. 3 & 6), it was shown that 3D perfusable microvessel networks are fully grown in 5-7 days through self-assembly.

In some embodiments, an exemplary device 100 comprising an in vitro tumor model (FIG. 3) is tissue engineered (e.g., formed within central chamber 104) with a central immunocompetent PDAC niche (FIG. 3C) comprising PDAC organoids, vasculature, cancer-associated fibroblasts (CAFs), niche immune cells (tumor-associated macrophages (TAMs), Treg cells, and myeloid-derived suppressor cells (MDSCs)), and an adjacent vascular network (FIG. 3C green) to serve as the pathway for nutrients, drugs and cells transport. In some embodiments, device 100 is fabricated with different culture compartments (e.g., first inner region 110 and first outer region 112) that are segregated by regularly spaced PDMS micropillars (e.g., plurality of micropillars 108) that restrict a cell-embedded hydrogel to simulate the in vivo architecture of TME. In some embodiments, device 100 is fabricated with media reservoirs (e.g., formed by or within one or more openings 106) for long-term media supply and/or one or more microfluidic microenvironmental regulator units 120 for hypoxia, fluids, and IFP control that are integrated surrounding and on top of a tissue culture chamber (FIG. 7).

In some embodiments, to integrate, position or culture PDAC organoids on device 100, PDAC organoids ready to passage are harvested from plates, dissociated into single cells by TrypLE (Thermo Fisher), mixed with HUVECs and/or GFP-HUVECS, immune cells (TAMs, Tregs, MDSCs), and CAFs/PSCs in 3D ECM (fibrinogen and cell basement membrane (ATCC)/Matrigel (Corning)/Cultrex Reduced Growth Factor Basement Membrane Extract, Type R1 (R&D Systems), and loaded in the first inner region 110. In some embodiments, the 3D ECM comprises fibrinogen and cell basement membrane from ATCC mixed in a ratio of 1:1. In some embodiments, culture media is optimized from a mixture of PDAC organoids medium and EGM-2 (Lonza) in a ratio of 1:1-1:5 with 50 ng/mL VEGF. In some embodiments, Y-27632 (10 μM, ATCC) is added in the medium for the first 3 days to increase the survival of PDOs on the chip device. After 5-7 days of tumor-stromal-immune interactions, a vascularized PDAC organoids-on-a-chip device 100 is fully developed (FIG. 3C).

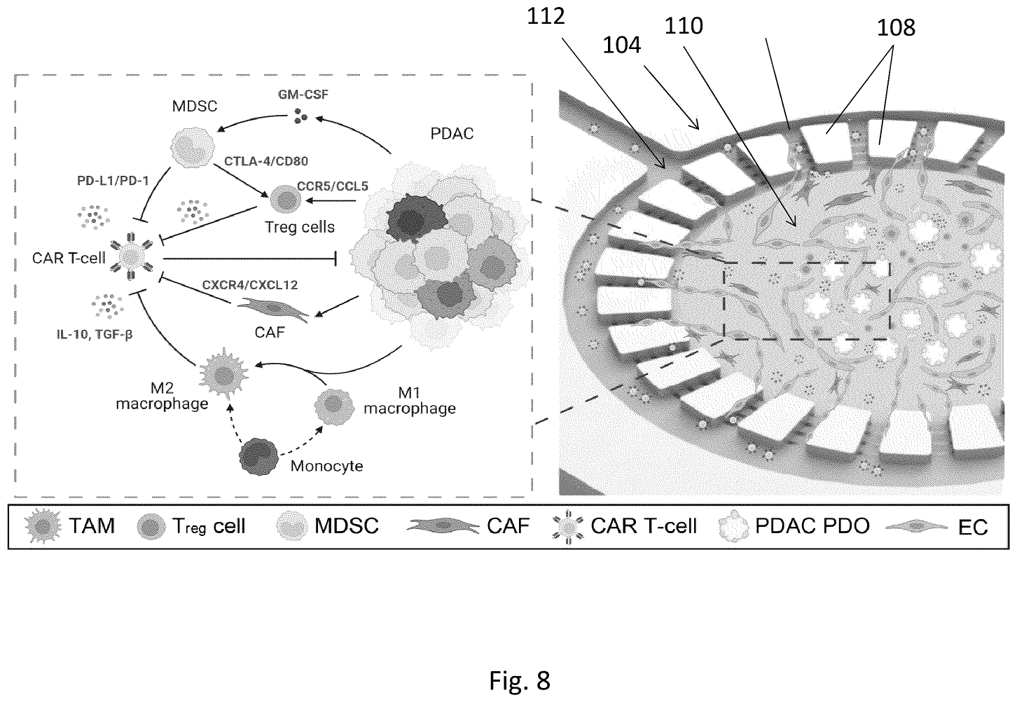

In some embodiments, in order to create a physiologically relevant immune niche, niche immune cells (e.g., TAMs, Treg cells, and MDSCs) are premixed with PDOs and stromal cells in 3D ECM, loaded in inner region 110 of device 100, and co-cultured for 5-7 days until the establishment of the immune and vascularized stromal niche is formed together on device 100 (FIGS. 8 & 9). In some embodiments, device 100 comprises or is formed with or using monocytes isolated by PDAC patients or healthy donors' peripheral blood mononuclear cells (PBMCs, iXCells Biotechnologies) using Human Pan Monocyte Isolation Kit (Biolegend) and treated with 25 ng/mL MCSF (R&D Systems) for 7 days to mimic CD14⁺CD68⁺ TAMs. In some embodiments, device 100 comprises or is formed with or using CD4⁺ T cells isolated using Human Naive CD4⁺ T Cell Isolation Kit (R&D Systems) and differentiated into CD4⁺ CD25⁺ FOXP3⁺ Treg cells by Human Treg Cell Differentiation Kit (R&D Systems). In some embodiments, device 100 comprises or is formed with or using CD11b⁺CD14⁻CD15⁺ MDSCs isolated using the Cell Isolation Kits (Miltenyi Biotec). In some embodiments, device 100 comprises or is formed with or using immune and tumor cells tuned with different ratios (e.g., normal: 5%-10%, cancerous: 20%-40% TAMs) and densities based on primary diagnostic tissue data to reflect patient pathophysiology.

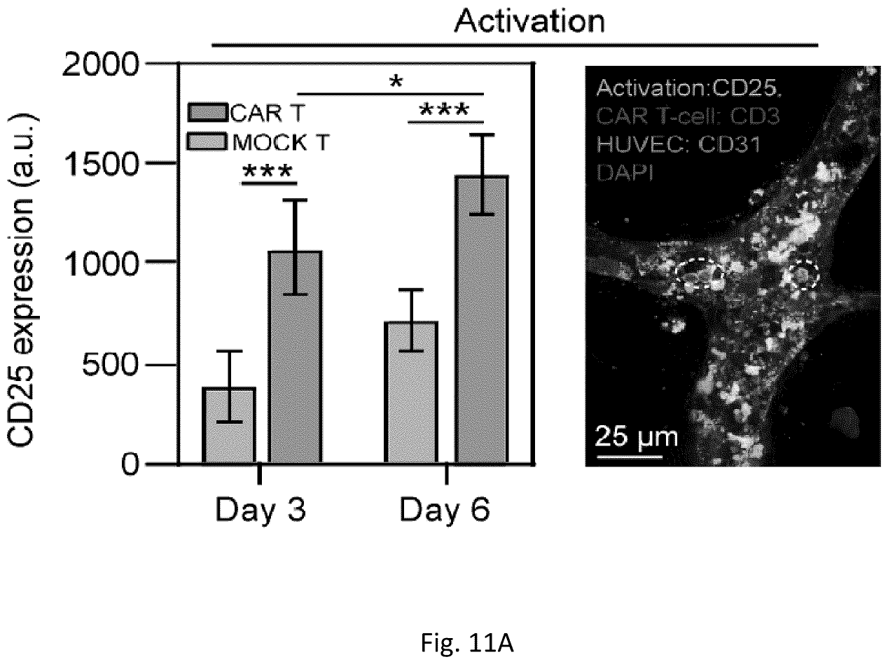

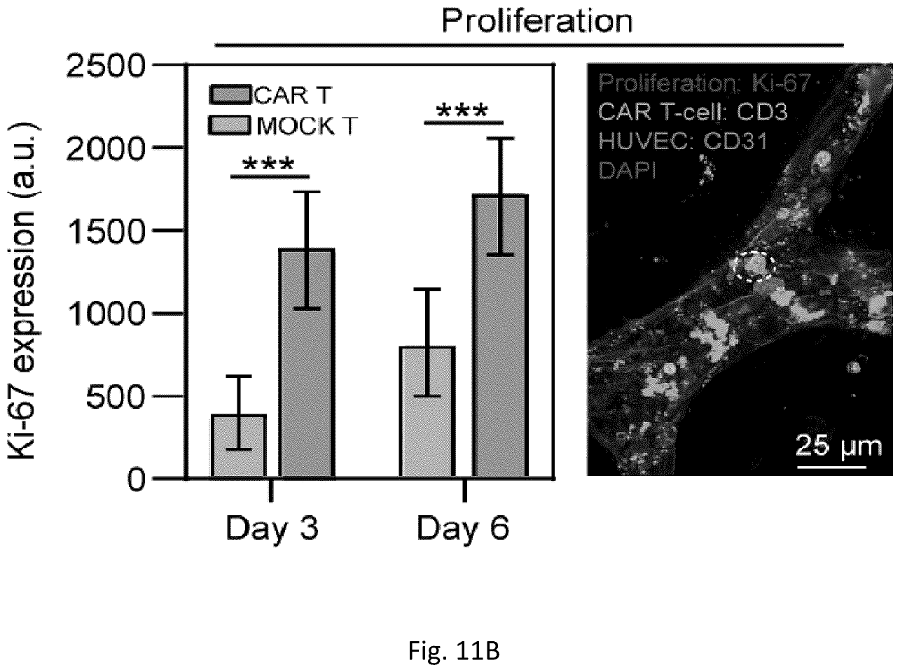

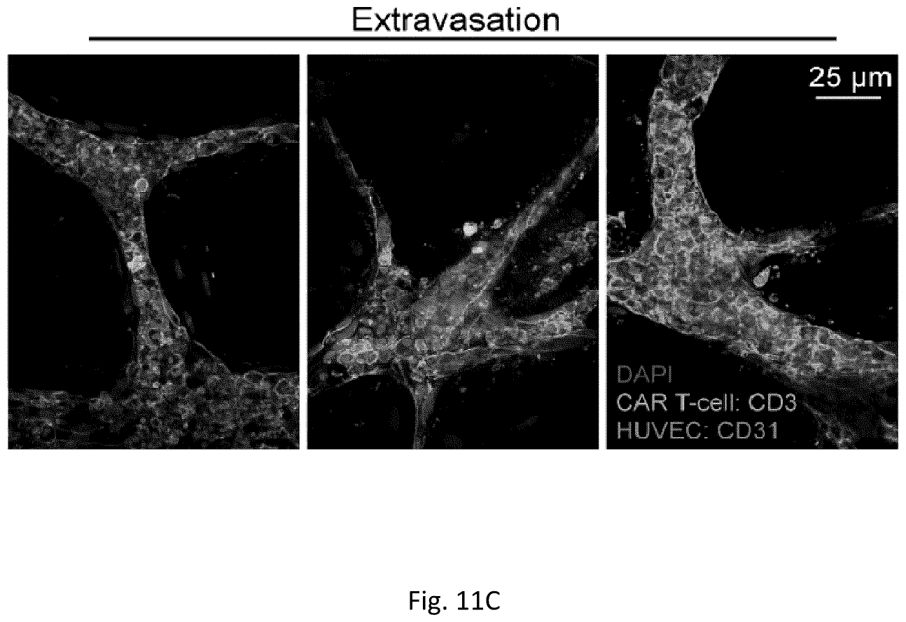

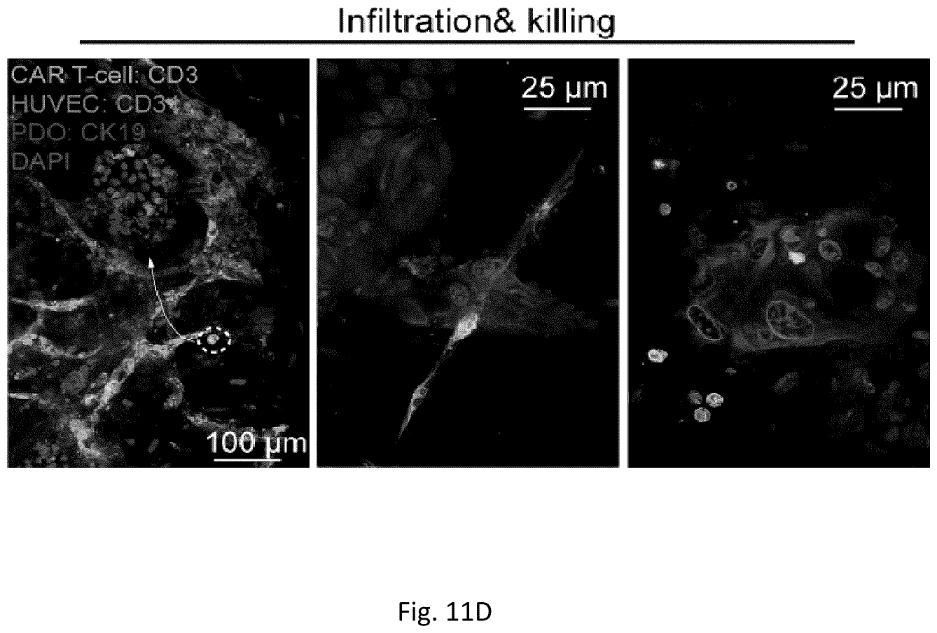

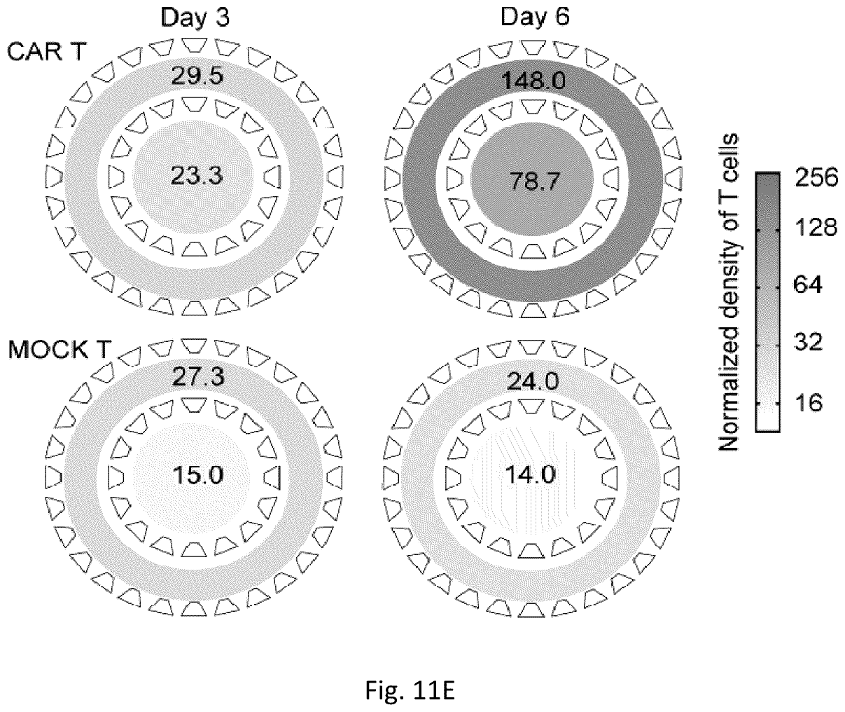

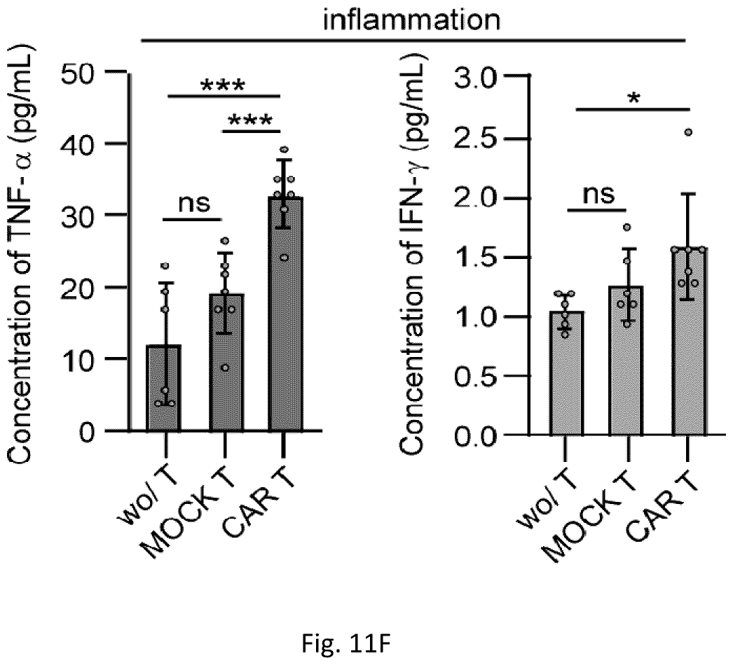

In some embodiments, in order to model the CAR T-cell therapy on an exemplary device 100, CAR T-cells are infused into the 3D perfusable microvessel (i.e. central chamber 104) in various densities (e.g., 2-5×10⁶ cells/ml) and ratios (e.g., CAR T-cell:tumor cell=1:10-1:1) and monitored, in real time, of their functional responses in the PDAC TME (FIG. 11). In some embodiments, device 100 comprises or is formed with or using CAR T-cells (ProMab Biotechnologies) targeting different antigens (e.g., MSLN, HER2, EGFR, CEA, MUC1 and CD133) with different costimulatory domains (e.g., CD28 or 4-1BB domain) cultured in ImmunoCult™-XF T Cell Expansion Medium (STEMCELL) supplemented with 200 U/mL Recombinant Human IL-2 (PeproTech) for 2 days before use.

### Methods of Use

Aspects of the present invention relate to exemplary methods of use for an organoids-on-a-chip device (e.g., device 100). Overall, the disclosed device and methods present a new paradigm for “clinical trials on a chip” studies that can improve the efficacy of therapies in PDAC. In some embodiments, an exemplary patient-derived “PDAC organoids-on-a-chip” micro-physiological device (e.g., device 100) and method is applied to reconstitute patient-specific PDAC TMEs in vitro for therapy modeling and screening. In some embodiments, different therapies like chemotherapy, immune checkpoint inhibitors and chimeric antigen receptor (CAR) T-cell therapy can be tested and the responses accessed by the device and method, by which the therapeutic regimen can be screened, optimized and personalized. In some embodiments, the disclosed device and method enable real-time characterization of pathophysiological processes (e.g. cancer cells apoptosis, cytotoxic cells proliferation, activation and trafficking) at high spatiotemporal resolution.

Aspects of the present invention relate to a method of determining pancreatic disease treatment responsiveness, comprising the steps of providing an organoids-on-a-chip device (e.g., device 100), administering a pancreatic disease treatment to a central chamber (e.g., central chamber 104), and determining pancreatic disease treatment responsiveness based on a measured change in the central chamber. In some embodiments, the pancreatic disease treatment is a therapy selected from the group consisting of: anti-cancer drug, immunotherapy, chemotherapy, radiation therapy, chemoradiation therapy, and targeted therapy.

In some embodiments, the measured change comprises measuring a change in one or more parameters selected from the group consisting of: CAR T-cell infiltration frequency, speed, trajectory, and distance, expression levels of activation (CD25, CD154, CD69), proliferation (Ki67), cytotoxic function (interferon gamma, granzyme B and perforin) markers, relative frequencies and immunosuppressive function of TAM (CD163, Fizz-1, Arg1, CSF-1) and Treg cell (CD4⁺CD25⁺Foxp3⁺, CTLA-4), immunosuppressive cytokine profile (e.g., TGF-β, IL-10, CCL2), expression level of immune checkpoints markers (e.g. PD1/PDL1), the expression level of targeted antigens on tumor and the apoptosis rate of tumor cells.

In some embodiments, the treatment responsiveness is classified into one or more categories. In some embodiments, the treatment responsiveness is classified into three categories: remission, stable disease and progression. In some embodiments, remission is defined as the number and average diameter of the PDOs decrease by 50% (or apoptosis rate larger than 50%), progression is defined as the number and average diameter of the PDOs increase by 25%, and stable disease is defined as neither remission nor progression.

In some embodiments, the disclosed device and methods may be used to dissect subtype/patient-specific PDAC TME followed by therapies, testing, and screening, and may contribute to personalized therapy development. In some embodiments, the device and methods may be used with, and/or is compatible with high throughput biological assays (e.g. molecular, cellular, and histological characterizations) as well as in-depth genetic analyses (e.g. scRNA-seq).

In some embodiments, the disclosed device and methods may be used for CAR T-cell therapy screening. The disclosed device and methods present a unique precision immuno-oncology platform that recapitulates the patient-specific tumor pathophysiology and host immunity that allows for a rapid (2-3 weeks) and accurate assessment of novel CAR designs (antigens, costimulatory domains) for PDAC treatment.

In some embodiments, the disclosed device and methods may be used for monitoring CAR T-cell trafficking at high spatiotemporal resolution. The disclosed device and methods enable real-time monitoring of CAR T-cells trafficking dynamics to a solid tumor that involves numerous sequential steps that include transport in the vessel, extravasation into the TME, migration, recognition of the tumor cells, and tumor cell killing.

In some embodiments, the disclosed device and method may be used for PDAC TME modeling. In some embodiments, device 100 can retain the cellular heterogeneity and phenotype fidelity of original tumor. In some embodiments, the PDAC model on device 100 allows investigation of the immunomodulatory mechanisms in the PDAC TME underlying PDAC resistance to immunotherapies, and screens new TME-associated biomarkers and treatment regimens to improve patient response to immunotherapies.

In some embodiments, the disclosed device and methods may be used for personalized immunotherapy development. In some embodiments, device 100 and any associated methods can be incorporated into clinical trials and provide a new paradigm for “clinical trials on a chip” studies that can help screen new biomarkers and potential responders, and significantly accelerate the pace for developing personalized immunotherapeutic strategies for PDAC patients to improve clinical responses.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Pancreatic Cancer Organoids-On-a-Chip

### Current Challenges in Cancer Immunotherapy Modeling and Screening

Pancreatic ductal adenocarcinoma (PDAC), the most common type of pancreatic cancer, is highly malignant with overall 5-year survival rate around 10% [Siegel R L, Miller K D, Jemal A. Cancer statistics, 2020. CA: A Cancer Journal for Clinicians. 2020; 70(1):7-30]. PDAC is projected to be the 2nd leading cause of cancer related deaths by the year 2030 [Quante et al., Projections of cancer incidence and cancer-related deaths in Germany by 2020 and 2030. Cancer Medicine. 2016; 5(9):2649-56]. Current treatments like surgery, chemotherapy, and immune checkpoint inhibitors are only suitable to a small subset of patients with low response rate. Thus, there remains an urgent and unmet need to develop safe and effective therapies for PDAC patient treatment. Chimeric antigen receptor (CAR) T-cell therapy have been remarked as novel therapeutic strategies in pilot clinical trials for treating different tumors including PDAC. However, although CAR T-cell therapy has shown remarkable success in hematologic malignancies, solid tumors such as pancreatic cancer seemed to be intrinsically resistant to CAR T-cell therapy, which is largely attributed to its “immune cold” tumor microenvironment (TME). The tumor immunosuppression and microenvironmental heterogeneity are emerging as potential barriers in efficient CAR T-cell treatment.

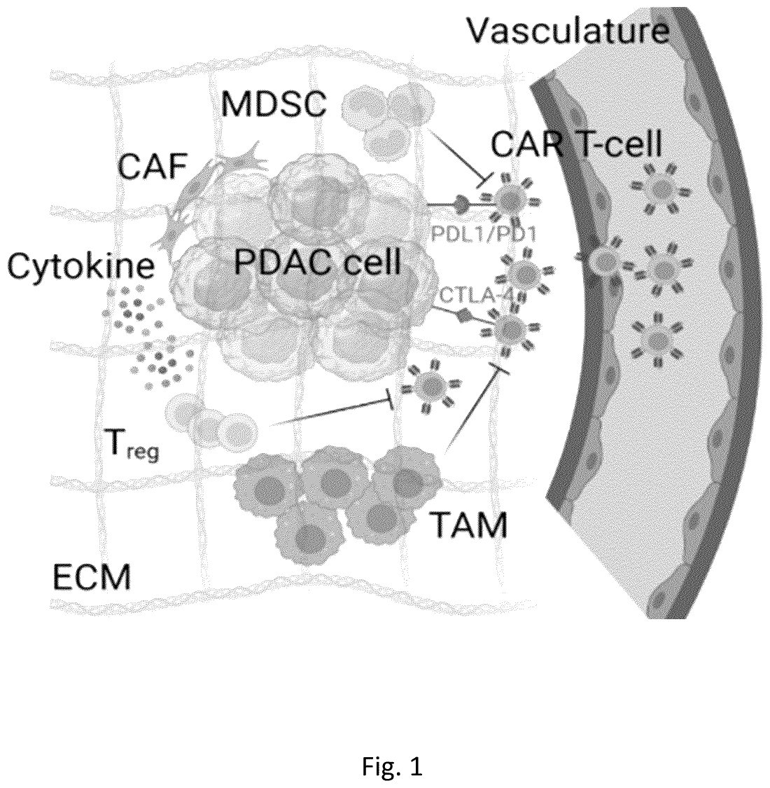

The PDAC TME (FIG. 1) is a “milieu” of distinct elements including altered extracellular matrix (ECM), fibroinflammatory stroma (cancer-associated fibroblasts, CAFs), infiltrating immunosuppressive cells (i.e., tumor-associated macrophages (TAMs), regulatory T (Treg) cells, and myeloid-derived suppressor cells (MDSCs)), and aberrant expression of checkpoint pathway proteins, immunosuppressive cytokines and molecules [Beatty et al., The biological underpinnings of therapeutic resistance in pancreatic cancer. Genes Dev. 2021; 35(13-14):940-62]. As an immune cold cancer, immune activities in PDAC TME result in repressive milieu facilitates multiple immunosuppression mechanisms against cytotoxic T lymphocyte (CTL) recruitment and functions in the TME that can translate to the suboptimal immunotherapy responses. For example, studies have found that myeloid cells, including macrophages and immature MDSCs, accumulate during the progression of pancreatic cancer and contribute to the treatment resistance to immunotherapies [Zhang et al., Myeloid cells are required for PD-1/PD-L1 checkpoint activation and the establishment of an immunosuppressive environment in pancreatic cancer. Gut. 2017; 66(1):124-36; Mitchem et al., Targeting tumor-infiltrating macrophages decreases tumor-initiating cells, relieves immunosuppression, and improves chemotherapeutic responses. Cancer Res. 2013; 73(3):1128-4]. These accumulated myeloid cells inhibit T-cell antitumor activity by inducing the expression of PD-L1 in tumor cells in an EGFR/MAPK-dependent manner [Zhang et al., Myeloid cells are required for PD-1/PD-L1 checkpoint activation and the establishment of an immunosuppressive environment in pancreatic cancer. Gut. 2017; 66(1):124-36]. In particular, the M2 inclined TAMs can induce the “immune cold” TME [Karamitopoulou E., The Tumor Microenvironment of Pancreatic Cancer. Cancers. 2020; 12(10):3076; Stromnes et al., Differential Effects of Depleting versus Programming Tumor-Associated Macrophages on Engineered T Cells in Pancreatic Ductal Adenocarcinoma. Cancer Immunol Res. 2019; 7(6):977-89] by secreting a panel of immunosuppressive cytokines like IL-6, IL-10 and TGF-β, inhibiting CTL immune activity, releasing PD-L1 and CTLA-4 to induce immune escape and promoting the recruitment, maturation, and differentiation of other immunosuppressive cells like Treg cells and MDSCs [Yang S, Liu Q, Liao Q. Tumor-Associated Macrophages in Pancreatic Ductal Adenocarcinoma: Origin, Polarization, Function, and Reprogramming. Front Cell Dev Biol. 2020; 8:607209].

In addition to immunosuppression, the unique stromal niche also limits the PDAC treatment efficacy. Activated by tumor cells, pancreatic stellate cells (PSCs) in stromal niche are transformed into CAFs. CAFs not only secrete excessive ECM containing collagen and hyaluronan, causing the desmoplasia stroma with dense barrier and preventing drug delivery and immune cells trafficking, but also promote tumor growth through biochemical cues like TGF-β and IL-6 [Vaish, Utpreksha, et al. “Cancer-associated fibroblasts in pancreatic ductal adenocarcinoma: an update on heterogeneity and therapeutic targeting.” International journal of molecular sciences 22.24 (2021): 13408; Perez V M, Kearney J F, Ych J J. The PDAC Extracellular Matrix: A Review of the ECM Protein Composition, Tumor Cell Interaction, and Therapeutic Strategies. Front Oncol. 2021; 11:751311]. Stromal niche factors like high interstitial flow pressure (IFP) produced through ECM and TGF-β receptor signaling pathway mediating vessel-tumor interactions create an inherently poorly vascularized PDAC TME [Nguyen et al., A biomimetic pancreatic cancer on-chip reveals endothelial ablation via ALK7 signaling. Science Advances. 5(8): caav6789; Provenzano et al., Enzymatic targeting of the stroma ablates physical barriers to treatment of pancreatic ductal adenocarcinoma. Cancer Cell. 2012; 21(3):418-29], which has been demonstrated by tumor samples from patients and mouse models [Craven K E, Gore J, Korc M. Overview of pre-clinical and clinical studies targeting angiogenesis in pancreatic ductal adenocarcinoma. Cancer Lett. 2016; 381(1):201-10; Olive K P et al., Inhibition of Hedgehog Signaling Enhances Delivery of Chemotherapy in a Mouse Model of Pancreatic Cancer. Science. 2009; 324(5933):1457-61]. Hypovascularity in PDAC stroma niche with collapsed vasculature and poor perfusion ultimately impairs the infiltration of CAR T-cells [Akce et al., The Potential of CAR T Cell Therapy in Pancreatic Cancer. Frontiers in Immunology. 2018; 9]. Other extracellular cues like hypoxia, resulting from the desmoplasia niche, are strongly associated with the immunosuppression and thus poor patient prognosis in PDAC [Provenzano et al., Enzymatic targeting of the stroma ablates physical barriers to treatment of pancreatic ductal adenocarcinoma. Cancer Cell. 2012; 21(3):418-29; Tao J et al., Targeting hypoxic tumor microenvironment in pancreatic cancer. Journal of Hematology & Oncology. 2021; 14(1):14; Yamasaki A, Yanai K, Onishi H. Hypoxia and pancreatic ductal adenocarcinoma. Cancer Lett. 2020; 484:9-15]. Together, the tumor-promoting CAFs, abnormal vasculature, dense ECM, high IFP and hypoxic signatures in stromal niche may aggravate TME immunosuppression and thwart CAR T-cell extravasation, thus permitting tumor escape from immune surveillance [Hao et al., Metabolic reprogramming due to hypoxia in pancreatic cancer: Implications for tumor formation, immunity, and more. Biomedicine & Pharmacotherapy. 2021; 141:111798].

PDAC is a genetically heterogeneous disease and can be classified into two major transcriptional subtypes based on the molecular signature of PDAC: a poorly differentiated aggressive basal-like subtype and a classical subtype with better prognosis [Xu Z et al., Clinical Impact of Molecular Subtyping of Pancreatic Cancer. Front Cell Dev Biol. 2021; 9:743908; Wood et al., Pancreatic Cancer: Pathogenesis, Screening, Diagnosis, and Treatment. Gastroenterology. 2022; 163(2):386-402.e1]. Basal-like subtype demonstrates an “immune escape” mechanism with a more hostile TME richer in M2-polarized TAMs and Treg cells and poor in effector T cells [Bailey P, et al., Australian Pancreatic Cancer Genome I. Genomic analyses identify molecular subtypes of pancreatic cancer. Nature. 2016; 531(7592):47-52; Facciabene A, Motz G T, Coukos G. T-Regulatory Cells: Key Players in Tumor Immune Escape and Angiogenesis. Cancer Research. 2012; 72(9):2162-71]. However, classical subtype is with an “immune rich” and cytotoxic TME, characterized with more effector T cells and M1-polarized TAMs, and lower level of Treg cells and M2-polarzied TAMs [Karamitopoulou E. Tumour microenvironment of pancreatic cancer: immune landscape is dictated by molecular and histopathological features. British Journal of Cancer. 2019; 121(1):5-14]. There is an urgent need to understand the highly heterogeneous and immunosuppressive TME and their effects on CAR T-cell therapy in a new patient-based immune-oncology model that mimics the clinical situation, and allows real-time testing of therapeutics.

There is a complete absence of reliable immuno-oncology models that can accurately dissect the patient-specific TME immune signatures and in-situ monitor the pathological process of PDAC with CAR T-cell treatment, which creates a major challenge in developing efficacious immunotherapy for solid tumors including PDAC. Conventional tumor tissue profiling and recent single-cell RNA sequencing (scRNA-seq) analysis capture only a snapshot of gene expressions at the time of the biopsy, and have failed so far to identify molecular biomarkers of CAR T-cell therapy. Animal and patient-derived xenografts (PDXs) models are considered the gold standard in current preclinical study of cancer therapies, yet they are not ideal for immunotherapy testing since they are either immunodeficient or differ from host immunity and fail to reflect the genetic heterogencity of primary tumors [Hingorani S R et al., Preinvasive and invasive ductal pancreatic cancer and its early detection in the mouse. Cancer Cell. 2003; 4(6):437-50; Garcia P L, Miller A L, Yoon K J. Patient-Derived Xenograft Models of Pancreatic Cancer: Overview and Comparison with Other Types of Models. Cancers (Basel). 2020; 12(5); Herreros-Villanueva et al., Mouse models of pancreatic cancer. World J Gastroenterol. 2012; 18(12):1286-94].

Patient-derived organoids (PDOs) have recently emerged as novel 3D culture systems that retain the heterogeneity and phenotype of original tumor with self-organization and self-renewal abilities [Larsen B M et al., A pan-cancer organoid platform for precision medicine. Cell Rep. 2021; 36(4):109429; Boj S F et al., Organoid models of human and mouse ductal pancreatic cancer. Cell. 2015; 160(1-2):324-38], however, several challenges exist in the production, control, and analysis of organoids, as well as modeling immune responses and TMEs. For example, 1) Microenvironmental control of organoids. PDOs mostly suffer from the drawback of not preserving the context of patient's immune and organotypic tumor architecture and pathophysiology (i.e., tumor vasculature, hypoxia, and physiological perfusion). As a result, the functional responses of effector T-cell and the dynamic tumor-immune-stroma interactions are not well characterized in patients' TMEs during treatments. This is a particular problem for the testing of CAR T-cell therapy. 2) Control of the biochemical and extracellular microenvironment. One of the biggest obstacles in growing organoids is the restricted nutrient supply, gas exchange and waste removal at the interior of the organoids and precise control over extracellular cues such as ECM, hypoxia, physiological fluid flow and IFP in TMEs. 3) Precision and reproducibility. In contrast to the highly reproducible process of organogenesis in vivo, organoids develop with substantial variability in size, structural organization, functional capacity, gene expression due to uncontrolled culture and niche factors. This inter-organoid variability has been identified as a major issue that limits the potential of organoid technology high-throughput therapy screening.

Microengineered 3D tumor models with microfluidics and organ-on-a-chip technologies have recently shown new approaches that may tackle these challenges associated with in vivo PDXs and in vitro PDOs [Ma et al., Organ-on-a-Chip: A New Paradigm for Drug Development. Trends Pharmacol Sci. 2021; 42(2):119-33]. By integrating living human cells within microengineered yet physiologically relevant microenvironments, microfluidic organ-on-a-chip technologies have enabled the recapitulation of the major functions and architectures of microscale human tissue, including tumor pathophysiology. In particular, these organ-on-a-chip systems provide for a controllable and reproducible 3D tissue culture module, allowing emulation of the microvascular network, control of cell-cell and cell-ECM contact, control of nutrient supply, and control of biochemical and biophysical conditions in a dynamic microenvironment.

However, in terms of cellular heterogeneity and phenotypic fidelity, current cell line-based organ-on-a-chip systems remain inferior to organoids [Takebe T, Zhang B, Radisic M. Synergistic Engineering: Organoids Meet Organs-on-a-Chip. Cell Stem Cell. 2017; 21(3):297-300], which could be complemented with organoids derived from patient cells. Most importantly, organ-on-a-chip based immune-oncology model still in its infancy. There are no existing patient-derived systems that can model CAR T-cell therapy in TME with the unique and complex hypovascularized, hypoxic, and immunosuppressive TME signatures.

Based on above, there is a critical demand for an accurate, real-time, and modular methodology to understand the highly heterogeneous and immunosuppressive TME, and to test and screen promising therapies mimicking the clinical situation. Organ-on-a-chip and PDOs represent two fundamentally different yet complementary approaches toward the same goal of recapitulating the complexity of human organs in vitro. Yet, Organ-on-a-chip or PDOs alone have limited capacity to meet the broad range of needs that arise in PDAC therapy and TME modeling. Therefore, disclosed is a strategic integration of the two emerging technologies, i.e., integrating PDAC PDOs on a vascularized TME chip with biomimetic stromal and immune niches, to address each approach's limitations and synergistically combine the best features of each approach to provide a more powerful in vitro technology for PDAC therapy modeling and screening.

The disclosed studies have developed 3D microfluidic in vitro tumor models (termed ‘Cancer-on-a-chip’) in order to replicate TMEs on-chips, study personalized chemo-, immuno- and combination therapies for cancer patients [Witkowski M T et al., NUDT21 limits CD19 levels through alternative mRNA polyadenylation in B cell acute lymphoblastic leukemia. Nature Immunology. 2022; 23(10):1424-32; Ma C et al., Leukemia-on-a-chip: Dissecting the chemoresistance mechanisms in B cell acute lymphoblastic leukemia bone marrow niche. Science Advances. 6(44):eaba5536; Witkowski M T et al., Extensive Remodeling of the Immune Microenvironment in B Cell Acute Lymphoblastic Leukemia. Cancer Cell. 2020; 37(6):867-82.e12; Cui X et al., Dissecting the immunosuppressive tumor microenvironments in Glioblastoma-on-a-Chip for optimized PD-1 immunotherapy. eLife. 2020; 9:e52253]. For example, a ‘Glioblastoma-on-a-Chip’ brain tumor model was engineered to dissect the mechanisms of resistance to anti-PD-1 immunotherapy [Cui X et al., Dissecting the immunosuppressive tumor microenvironments in Glioblastoma-on-a-Chip for optimized PD-1 immunotherapy. eLife. 2020; 9:e52253]. A human ‘Leukemia-on-a-Chip’ model was also developed [Ma C et al., Leukemia-on-a-chip: Dissecting the chemoresistance mechanisms in B cell acute lymphoblastic leukemia bone marrow niche. Science Advances. 6(44):eaba5536], mimicking the in vivo leukemic bone marrow niche for a preclinical study of chemotherapy [Ma C et al., Leukemia-on-a-chip: Dissecting the chemoresistance mechanisms in B cell acute lymphoblastic leukemia bone marrow niche. Science Advances. 6(44):eaba5536] and CAR T-cell therapy [Witkowski M T et al., NUDT21 limits CD19 levels through alternative mRNA polyadenylation in B cell acute lymphoblastic leukemia. Nature Immunology. 2022; 23(10):1424-32], which allows real-time monitoring of the CD19 CAR T-cell extravasation, activation, and killing activities.

The disclosed design is a patient-derived “PDAC Organoids-on-a-Chip” micro-physiological system formed by strategically integrating the state-of-the-art organoids and organ-on-a-chip technologies to accurately reconstitute the pathophysiology and heterogeneity of the PDAC TME in vitro, in order to study mechanisms of resistance, and assess, optimize and personalize different therapies. In some embodiments, the PDAC Organoids-on-a-Chip in vitro tumor model was tissue engineered with a central vascularized immunocompetent PDAC niche (center region, or first inner region 110) with PDAC patient-derived organoids (PDOs), vasculature, CAFs, niche immune cells (TAMs, Treg cells, and MDSCs), and an adjacent vascular network (outer ring, or first outer region 112) to serve as the pathway for nutrients, drugs and cells transport. In some embodiments, different culture compartments were segregated by regularly spaced PDMS micropillars (e.g., plurality of micropillars 108) that restrict cell-embedded hydrogel to simulate the in vivo architecture of TME. In some embodiments, media reservoirs for long-term media supply were surrounding of the tissue culture chamber.

The disclosed design includes the key stroma and immune niche factors in PDAC TME with controllable biological features (e.g. cell type and density, ECM components), and maintains the original patient-specific features in PDOs with high pathological relevance. In some embodiments, the chip enables real-time characterization of pathophysiological processes (e.g. cancer cells apoptosis, cytotoxic cells proliferation, activation and trafficking) at high spatiotemporal resolution. The disclosed chip is compatible with high throughput biological assays (e.g. molecular, cellular, and histological characterizations) as well as in-depth genetic analyses (e.g. scRNA-seq). The chip can dissect subtype/patient-specific PDAC TME followed with therapies testing and screening, and finally contribute to personalized therapy development.

The patient-derived “PDAC organoids-on-a-chip” micro-physiological system was applied to reconstitute patient-specific PDAC TMEs in vitro for therapy modeling and screening. Different therapies like chemotherapy, immune checkpoint inhibitors and chimeric antigen receptor (CAR) T-cell therapy were tested and the responses assessed on chip, by which the therapeutic regimen were screened, optimized and personalized. Overall, the disclosed chip demonstrated a new paradigm for “clinical trials on a chip” study that can improve the efficacy of therapies in PDAC.

In some embodiments, the disclosed device comprises third-party biological materials. PDAC patient-derived organoids (PDOs) line PDM-40, PDM-41 may be sourced from Human Cancer Models Initiative of ATCC, PDO lines 242566, 485368, 671287, 868679, 982776 from NCI PDMR, PDOs lines NYU318, NYU341, NYU421, NYU429, NYU455 from NYU Langone Health, or PDOs from any institutional cancer center. PDOs are cultured in Cell Basement Membrane (ATCC). The organoids culture medium is PancreaCult Organoid Media (Human) (STEMCELL), and/or the components of the one or more organoid culture mediums comprise Advanced DMEM:F12 (Thermo Fisher), HEPES (Thermo Fisher), B-27 supplement (Thermo Fisher), GlutaMAX™ (Thermo Fisher), noggin (Bio-techne), EGF (Bio-techne), Gastrin (Bio-techne), FGF-10 (Bio-techne), A83-01 (Bio-techne), nicotinamide (Sigma), N-acetyl cysteine (Sigma), RSPO-1 conditioned medium (Sigma) and Wnt-3A conditioned medium (MBL) and Y27632 (Y27632).

Pancreatic Stellate Cells (PSCs, a major source of CAFs, ScienCell) were used, cultured in Stellate Cell Medium (SteCM, ScienCell), patient-derived CAFs cultured in RPMI 1640 medium (Thermo Fisher), primary human umbilical vein endothelial cells (HUVECs)/primary GFP expressing human umbilical vein endothelial cells (GFP-HUVECs, Angioproteomie) cultured in endothelial cells growth medium (EGM-2, Lonza) and vascular niche supporting cells (normal human lung fibroblasts, NHLFs, Lonza) cultured in fibroblast cells growth medium (FGM-2, Lonza) mixed in a fibrinogen hydrogel (Sigma).

Monocytes were isolated from patient or healthy donor-derived peripheral blood mononuclear cells (PBMCs, iXCells Biotechnologies) using Human Pan Monocyte Isolation Kit (Biolegend) and treated with 25 ng/ml MCSF (R&D Systems) for 7 days to mimic CD14⁺CD68⁺ TAMs. CD4⁺ T cells are isolated using Human Naive CD4⁺ T Cell (R&D Systems) and differentiated into CD4⁺ CD25⁺ FOXP3⁺ Treg cells by Human Treg Cell Differentiation Kit (R&D Systems). CD11b⁺CD14⁻CD15⁺ MDSCs were isolated using the Cell Isolation Kits (Miltenyi Biotec).

### A Biomimetic PDAC TME Model by Integrating PDAC Organoids and Organ-On-a-Chip

The PDAC Organoids-on-a-Chip in vitro tumor model (FIG. 3) was tissue engineered with a central vascularized immunocompetent PDAC niche (center region; FIG. 3C) with PDAC organoids, 3D vascular network, CAFs, niche immune cells (TAMs, Treg cells, and MDSCs), and an adjacent vascular region (outer ring; FIG. 3C green) to serve as the interface of tumor-stroma-immune interactions. Different culture compartments were segregated by regularly spaced PDMS micropillars that restrict cell-embedded hydrogel to simulate the in vivo architecture of TME. Media reservoirs for long-term media supply and microfluidic microenvironmental regulator units for hypoxia, fluids, and IFP control were integrated surrounding and on top of the tissue culture chamber (FIG. 7).

### PDO Culture and Characterization

Twelve (12) lines of PDOs were utilized covering major molecular subtypes (basal-like and classical) for the validation of the patient-derived PDAC chips. PDAC PDOs (FIG. 3A) from Human Cancer Models Initiative of ATCC (PDM-41) were successfully cultured and experiments were conducted. A large bank of pancreatic cancer organoids were created from patients with different subtypes, all stages of disease and accompanying extensive clinical data. This growing bank was used to validate the proposed PDAC organoids-on-a-chip model. PDAC organoids were mixed in 3D ECM (Cell Basement Membrane, ATCC) and cultured in 12-well plates. PDAC organoids medium is supplied and refreshed every 2-3 days. The organoids culture medium is PancreaCult Organoid Media (Human) (STEMCELL), and/or the components of organoid medium were Advanced DMEM:F12, HEPES, B-27 supplement, GlutaMAX™, noggin, EGF, Gastrin, FGF-10, A83-01, nicotinamide, N-acetyl cysteine, RSPO-1 conditioned medium and Wnt-3A conditioned medium. Y27632 (a ROCK signaling pathway inhibitor) is added in organoid medium for the first 2-3 days to increase the survival of organoids. Organoids were subcultured (density of 2.5-3×10⁶ cells/mL) in 14 days and used within 10 passages. Based on transcriptomic profiling [Driehuis E et al., Pancreatic cancer organoids recapitulate disease and allow personalized drug screening. Proc Natl Acad Sci USA. 2019; 116(52):26580-90], PDOS were stratified into either invasive basal-like or indolent classical subtype [Moffitt R A et al., Virtual microdissection identifies distinct tumor- and stroma-specific subtypes of pancreatic ductal adenocarcinoma. Nature Genetics. 2015; 47(10):1168-78].

### Microfabrication

The microfluidic chip was fabricated using standard PDMS soft lithography technique [Cui X et al., Dissecting the immunosuppressive tumor microenvironments in Glioblastoma-on-a-Chip for optimized PD-1 immunotherapy. Elife. 2020; 9:e52253; Ma C et al., Leukemia-on-a-chip: Dissecting the chemoresistance mechanisms in B cell acute lymphoblastic leukemia bone marrow niche. Sci Adv. 2020; 6(44)], then PDAC organoids and different niche cells were compartmentalized on the chip in different culture compartments following a multiple-step loading and self-assembly protocol [Ma C et al., Leukemia-on-a-chip: Dissecting the chemoresistance mechanisms in B cell acute lymphoblastic leukemia bone marrow niche. Sci Adv. 2020; 6(44)].

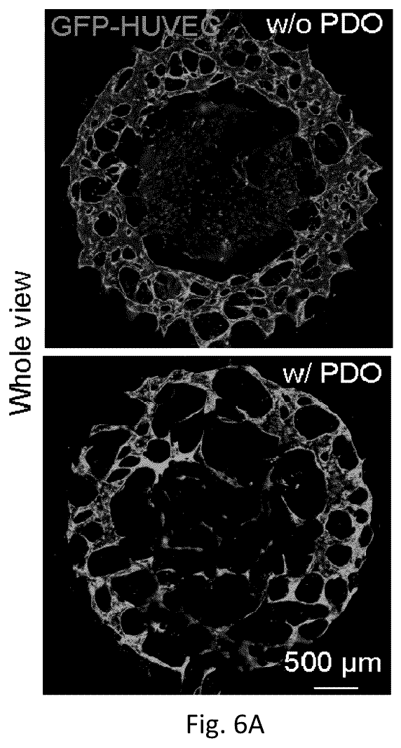

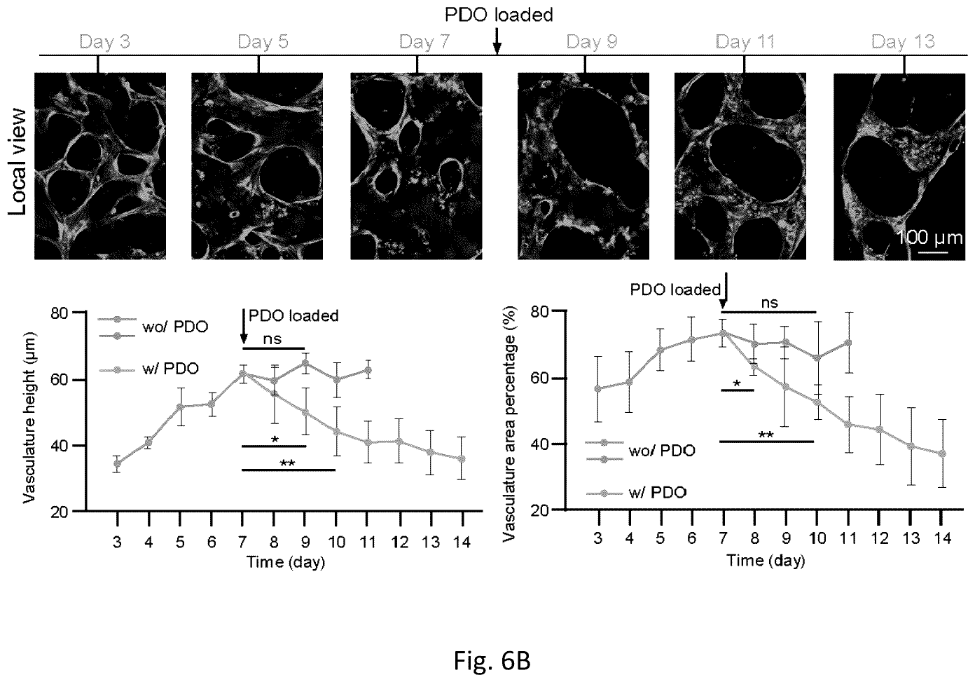

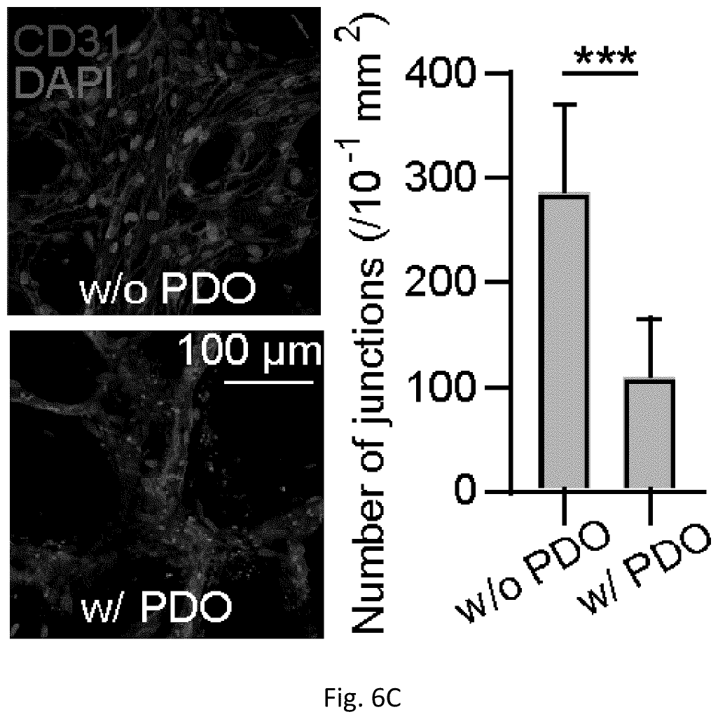

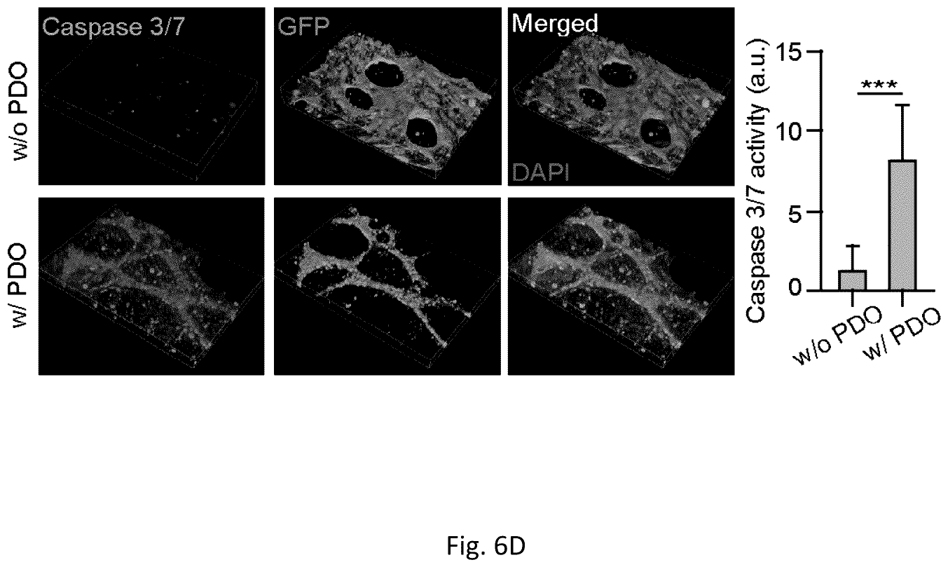

To recapitulate the vascularized stromal niche (0-7 days), human umbilical vein endothelial cells (HUVECs, Lonza)/primary GFP expressing human umbilical vein endothelial cells (GFP-HUVECs, Angioproteomie) and vascular niche supporting cells (normal human lung fibroblasts, NHLFs, Lonza) with optimized seeding density for each cell type (e.g. 10⁶-10⁷ cells/ml) were loaded in a fibrinogen hydrogel (3 mg/mL, Sigma) in the outer ring (first outer region 112) of the microfluidic chip (FIGS. 3C & 6A). EGM-2 (Lonza) medium with 50 ng/mL VEGF has been validated to support the growth of stromal cells on chip. Based on the disclosed study (FIGS. 3 & 6), it was expected that 3D perfusable microvessel networks would be fully grown in 5-7 days through self-assembly. Before PDO loading, the vascular network should grow maturely in the first 7 days occupying >75% area of the outer ring of the device with average diameter >100 μm and height >70 μm (FIG. 6A). The intactness of the vessels were firstly assessed by quantifying the junctions formed by HUVECs on vascular walls (staining CD31, FIG. 6C) and the average number of junctions is expected to be >90 per 10⁻¹ mm². Fluorescent dextran was applied to quantify the permeability of the vasculature [Hu Z et al., Vascularized Tumor Spheroid-on-a-Chip Model Verifies Synergistic Vasoprotective and Chemotherapeutic Effects. ACS Biomaterials Science & Engineering. 2022; 8(3):1215-25]. Permeability <4×10⁻¹¹ m²/s indicates a low permeability and high intactness.

To integrate PDAC organoids on the chip, PDAC organoids were loaded in the center chamber of the chip and then vascularized PDAC PDOs to form the whole 3D tumor niche in 5 days (FIG. 3C). PDAC organoids ready to passage were harvested from plates, dissociated into single cells by TrypLE (Thermo Fisher), and mixed with HUVECs, immune cells (TAMs, Tregs, MDSCs), and CAFs/PSCs in 3D ECM (fibrinogen and cell basement membrane from ATCC mixed in a ratio of 1:1) under an optimized density (e.g., 10⁶-10⁷ cells/ml). Culture media was optimized from a mixture of PDAC organoids medium (as indicated in PDO culture) and EGM-2 (Lonza) in a ratio of 1:1 with 50 ng/mL VEGF. Y27632 (10 μM, ATCC) was added in the medium for the first 3 days to increase the survival of PDOs on the chip. After 5-7 days of tumor-stromal-immune interactions, a vascularized PDAC organoids-on-a-chip was fully developed (FIG. 3C).

To create a physiologically relevant immune niche, niche immune cells (e.g., TAMs, Treg cells, and MDSCs) were premixed with PDOs and stromal cells in 3D ECM, loaded in the center chamber of the chip, and co-cultured for 5-7 days until the establishment of the immune and vascularized stromal niche together on chip (FIGS. 8 & 9). Specifically, Monocytes were isolated by PDAC patients or healthy donors' peripheral blood mononuclear cells (PBMCs, iXCells Biotechnologies) using Human Pan Monocyte Isolation Kit (Biolegend) and treated with 25 ng/ml MCSF (R&D Systems) for 7 days to mimic CD14⁺CD68⁺ TAMs. CD4⁺ T cells were isolated using Human Naive CD4⁺ T Cell isolation kit (R&D Systems) and differentiated into CD4⁺ CD25⁺ FOXP3⁺ Treg cells by Human Treg Cell Differentiation Kit (R&D Systems). CD11b⁺CD14⁻CD15⁺ MDSCs were isolated using the Cell Isolation Kits (Miltenyi Biotec). Loaded immune and tumor cells were tuned with different ratios (e.g., normal: 5%-10%, cancerous: 20%-40% TAMs) and densities based on primary diagnostic tissue data collected to reflect patient pathophysiology.

### Engineering a Patient-Derived In Vitro PDAC Organoids-On-a-Chip Model to Reconstitute the In Vivo Stromal Niche of the PDAC TME

Stromal characteristics such as dense ECM, poor vasculature, hypoxia, and high IFP are critical features of PDAC TME. Yet how these cellular and non-cellular components in the stromal niche regulate PDAC invasion, progression, and therapy responses remain unclear. Current in vivo and in vitro models fall short on how to investigate such cues controllably and dynamically. PDOs have recently emerged as novel 3D culture systems that can retain the heterogeneity and phenotype of original tumor, however, lack key stromal features and control of the biochemical and extracellular microenvironment. Herein, the state-of-the-art organoids and organ-on-a-chip technologies were strategically integrated to reconstruct PDAC PDOs with key features of PDAC stromal niche like poor vasculature, dense stroma, CAFs-related signaling, hypoxia and high IFP on chip.

### Integrating and Tuning Biochemical and Biophysical Cues in the Tumor Niche

Extracellular cues in the PDAC TME, such as gradients of oxygen and flow perfusion, were modulated by and in turn promote PDAC progression. For example, hypoxia is a prominent feature of PDAC TME, resulting from the abundant stromal cells and ECM and hypovasculature [Tao J et al., Targeting hypoxic tumor microenvironment in pancreatic cancer. Journal of Hematology & Oncology. 2021; 14(1):14] and lead to tumor proliferation and invasion [Yamasaki A, Yanai K, Onishi H. Hypoxia and pancreatic ductal adenocarcinoma. Cancer Lett. 2020; 484:9-15]. Hypoxia is also demonstrated as a strong inducer of IL1α which promotes the formation of inflammatory CAF in PDAC [Mello A M et al., Hypoxia promotes an inflammatory phenotype of fibroblasts in pancreatic cancer. Oncogenesis. 2022; 11(1):56]. Elevated IFP, as a result of dense stroma in PDAC, can lead to vasculature collapses thus hinder drug delivery and T cell infiltration [DuFort Christopher C et al., Interstitial Pressure in Pancreatic Ductal Adenocarcinoma Is Dominated by a Gel-Fluid Phase. Biophysical Journal. 2016; 110(9):2106-19; Akce et al., The Potential of CAR T Cell Therapy in Pancreatic Cancer. Front Immunol. 2018; 9:2166]. However, precise control over extracellular cues in the TME is largely lacking in current methods including PDOs. To address this limitation, microfluidic microenvironmental regulator units were integrated on chip for hypoxia, fluids, and IFP control in tumor niche (FIG. 7).

The microfluidic culture unit integrated an oxygen gas regulator that supported tunable oxygenation conditions (0-20%; 0-42 ppm) and a hydrostatic pressure gradient across the gel region controlled by a digitally-controllable pressure pump (Elveflow) to recreate the pathologically-relevant hypoxia and IFP in the disclosed tumor niche model (FIGS. 7A & 7B). To cover the physiologically-relevant ranges of hypoxia and IFP in the TME, local oxygen concentrations were controlled in the range of 0.1˜8% with 0.5% accuracy, IFP was controlled in the range of 0-150 mmHg with a resolution of 0.3% full scale (FS) and pressure stability of 0.5% FS. Specifically, the oxygenator includes pairs of microchannels fabricated in the gas layer for mixing oxygen and nitrogen to generate physiological (5-8% oxygen), pathophysiological (1-2% oxygen), and severe (0.1-1% oxygen) levels of hypoxia in tumors. To mimic different levels of IFP in the tumor, the pressure ranging from normal (˜30 mmHg) to malignant (˜100 mmHg) conditions was precisely controlled. The pressure flow is controlled by a digitally-controllable pressure pump in a range of 0-150 mmHg. The pressure gradient was validated by both numerical simulations with COMSOL Multiphysics and experiments with fluorescent microspheres (0.2 μm, Invitrogen). The disclosed microfluidic system permits long-term culture (1-2 weeks) to study the effects of hypoxia and IFP on tumor.

By tuning the hypoxia and IFP in physiological relevance, similar HIF-1α expression, vasculature growth and immunosuppression characteristics were recapitulated as the clinical tumor samples on chip. Besides, by adjusting these extracellular cues, it was confirmed whether they can cause the alternation of stroma niche. For example, the components and density of ECM were characterized, the variation of iCAF subpopulation and the secretion of IL1α was evaluated, and the evolution of vascular network was monitored. The ECM reorganization was examined in different levels of IFP by examining expressions of key ECM components including collagen, laminin, integrin, glycoproteins and HA, which influence immune cell infiltration and tumor growth [Perez V M, Kearney J F, Yeh J J. The PDAC Extracellular Matrix: A Review of the ECM Protein Composition, Tumor Cell Interaction, and Therapeutic Strategies. Front Oncol. 2021; 11:751311].

### Analytical Validation of the PDAC Organoids-On-a-Chip's Biomimicry to the In Vivo Niche

A high reproducibility was expected to be achieved as the microfluidic chip provides a well-controlled and organized growing environment with synthetic hydrogel with defined components as ECM [Ng S et al., Mechanically and chemically defined hydrogel matrices for patient-derived colorectal tumor organoid culture. Biomaterials. 2019; 219:119400; Gjorevski N et al., Designer matrices for intestinal stem cell and organoid culture. Nature. 2016; 539(7630):560-4] and growth factor supplements [Wensink G E et al., Patient-derived organoids as a predictive biomarker for treatment response in cancer patients. npj Precision Oncology. 2021; 5(1):30] for PDOs, thus increasing the uniformity of the PDO sizes, shape and distribution. Well grown PDAC organoids should have the average diameter >50 μm after 5 days and >90 μm with >90% viability after 9 days loaded on the chip. Hematoxylin and cosin (H&E) staining was applied as well to show the similarity of morphology between PDOs and tumors [Driehuis E et al., Pancreatic cancer organoids recapitulate disease and allow personalized drug screening. Proc Natl Acad Sci USA. 2019; 116(52):26580-90]. The transcriptional subtypes based on their subtype signature expressions proposed by Moffitt. et al [Moffitt R A et al., Virtual microdissection identifies distinct tumor- and stroma-specific subtypes of pancreatic ductal adenocarcinoma. Nature Genetics. 2015; 47(10):1168-78] were further validated by the immunostaining with canonical markers CK17 for basal-like subtype and GATA6 for classical subtype [Werba G et al., Single-cell RNA sequencing reveals the effects of chemotherapy on human pancreatic adenocarcinoma and its tumor microenvironment. Nature Communications. 2023; 14(1):797]. Moreover, cells from the device were retrieved, and scRNA-seq was performed to validate the concordance of the gene mutations between the on-chip niche and the tumor samples and the retainment of the heterogeneity and phenotype of original tumors on chip.

The patient-derived PDAC organoids-on-a-chip retained the cellular heterogeneity and phenotype fidelity of primary PDAC tumor, reduced inter-organoid variability, and faithfully recapitulated the in vivo pathophysiology of PDAC stromal niche. Given the possibility of low viability of PDOs in in vitro culture, the loading density of PDAC organoids may be increased to complement the loss, which was anticipated to not be more than 30%. To maintain a long-term culture, media was further optimized for different cell populations, and perfusion was tested if static culture failed to maintain cell function. The hypoxia and IFP was expected to be tuned to replicate the extracellular cues on chip to reflect the unique in vivo PDAC TME. Note that more severe hypoxia/IFP may compromise stromal cell viability, and thus, tumor maintenance. Therefore, optimal experimental hypoxia/IFP conditions was first screened to achieve >80% viability for all cells.

It was validated that the PDAC-organoids-on-a-chip recapitulate the in vivo immune niche of PDAC. It was also proven that stromal niche such as CAF-mediated desmoplasia and inflammatory response also influence the immunosuppressive PDAC TME. Although an immune niche fully derived from patient samples is the ideal condition, it should be noted that the availability of niche immune cells from patients may not be guaranteed. As an alternative, immune cells from PBMCs from healthy donors were adopted to validate the chip in the early phase. After establishing a prototype device with key immune cells, developing a fully patient derived PDAC-on-a-chip was then considered based on it using matched immune cells. Moreover, long-term culture of immune cells may also be challenging. The culture condition (e.g. cell density, medium and ECM components) was optimized and the disclosed results already suggest a successful culture of immune cells on chip for 7 days.

Establishment of biomimetic immunosuppressive niche was characterized as increased (>50%) ratios of immunosuppressive CD163⁺ M2 to immunoactive HLA-DR⁺ M1 TAMs, upregulation of immune checkpoints like CTLA-4, PD-1, TIM-3 and LAG-3 on Treg cells, PD-L1 and CD80 on MDSCs, and increased (>2-fold) secretion of immunosuppressive cytokines (e.g., TGF-β, IL-10, CCL2, IL-2). To confirm the biomimicry of the in vitro and in vivo immune niches, scRNA-seq on tumor tissue and PDAC niche on chip was adopted, and the among cell clusters and featured gene expressions were compared. The disclosed engineered TMEs were calibrated based on the primary diagnostic tissue data to better retain the in vivo features of tumor of specific patients or molecular subtypes.

Twelve (12) lines of PDOs (6 basal-like and 6 classical) were utilized to dissect the heterogeneity of immunosuppressive PDAC TMEs among different patients and molecular subtypes. It was expected for the chip to characterize 3 immune cell types (TAM, Treg and MDSCs) with >90% viability and single-cell solution of analysis. Distinct immunosuppression files were measured among different PDAC subtype-derived chips based on the forementioned multidimensional analysis (scRNA-seq, histology, cytokines, surface markers, immune checkpoints, etc.). It was aimed to establish an inter- and intra-assay reproducibility of 80% (Pearson correlation) at the lower limit of detection across all tested PDAC PDOs.

### Examining the Stromal Characteristics in the PDAC Chip

The disclosed study explored how activation of CAF remodels the stromal niche and promotes PDAC progression. PSCs activated by PDAC differentiate into different subtypes of CAFs [Norton J, Foster D, Chinta M, Titan A, Longaker M. Pancreatic Cancer Associated Fibroblasts (CAF): Under-Explored Target for Pancreatic Cancer Treatment. Cancers (Basel). 2020; 12(5)] and remodel the stromal niche to be hypovascularized with highly desmoplastic stroma as revealed by histological analysis [Feig et al., The Pancreas Cancer Microenvironment. Clinical Cancer Research. 2012; 18(16):4266-76]. First the activation of PSCs in the stromal niche was characterized through fibroblast activation protein (FAP). The activation of PSCs was compared between niches with or without PDOs to confirm the role of tumor niche in the generation of CAFs.

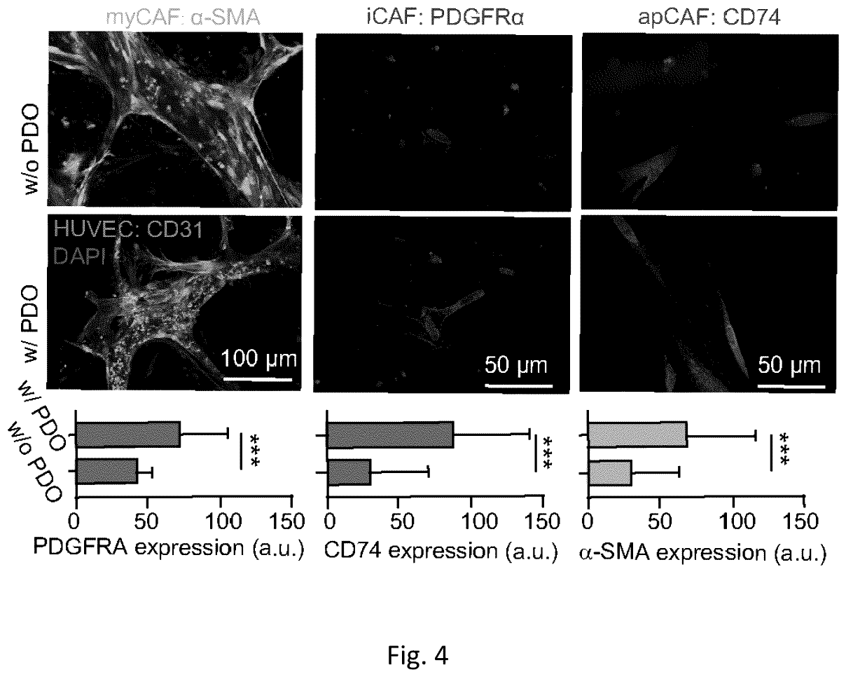

Next, the activations of different subtypes of CAFs by PDAC on chip were validated [Vaish U, Jain T, Are A C, Dudeja V. Cancer-Associated Fibroblasts in Pancreatic Ductal Adenocarcinoma: An Update on Heterogeneity and Therapeutic Targeting. Int J Mol Sci. 2021; 22(24)]. To validate the activations and distribution of different subtypes of CAFs on chip, different subtypes of CAFs were identified by biomarkers in the PDAC TME with significant difference compared to control groups: myCAFs highly expressed α-SMA and adjacent to PDOs; iCAFs was characterized by the secretion of IL-6, the expression of PDGFRα and a larger distance from PDOs; apCAFs expressed CD74 and MHC II. Among different subtypes of CAFs, myofibroblasts (myCAFs) enhance ECM protein production and induce desmoplasia in stromal niche [Feig et al., The Pancreas Cancer Microenvironment. Clinical Cancer Research. 2012; 18(16):4266-76]; inflammatory CAFs (iCAFs) secrete tumor-promoting inflammatory cytokines like IL-6 and IL-1 and mediate immunosuppression through CXCL12/CXCL4 axis, shows plasticity between myCAFs through JAK/STAT signaling pathway [Biffi Et al., IL1-Induced JAK/STAT Signaling Is Antagonized by TGFβ to Shape CAF Heterogeneity in Pancreatic Ductal Adenocarcinoma. Cancer Discovery. 2019; 9(2):282-301]; and antigen presenting CAFs (apCAFs) regulate tumor immunity by activating CD4⁺ T cells and might contribute to the immunosuppressive TME [Vaish U, Jain T, Are A C, Dudeja V. Cancer-Associated Fibroblasts in Pancreatic Ductal Adenocarcinoma: An Update on Heterogeneity and Therapeutic Targeting. Int J Mol Sci. 2021; 22(24) Norton JFDCMTALMPCAFU-ETIPCT. Cancers [Internet]. 2020; 125]. Different subtypes of CAFs were thus identified by the expression of biomarkers and distribution in the PDAC TME: myCAFs is α-SMAhigh, IL-6low and adjacent to PDOs; iCAFs is α-SMAlow, IL-6high and distant from PDOs; apCAFs express CD74 and MHC II (as shown in FIG. 4).

It was hypothesized that PDAC activates CAFs reciprocally and promotes tumor growth. To validate the this, PDAC PDOs on chip were observed under the microscope (Zeiss Primovert) daily and the diameters of PDOs were measured by ImageJ (NIH). In the presence of PSCs, the growth rate of PDAC PDOs was expected to accelerate on chip, indicating the key role of PSCs in facilitating PDAC progression.

### Explore the CAF-Mediated Desmoplasia in PDAC Stromal Niche

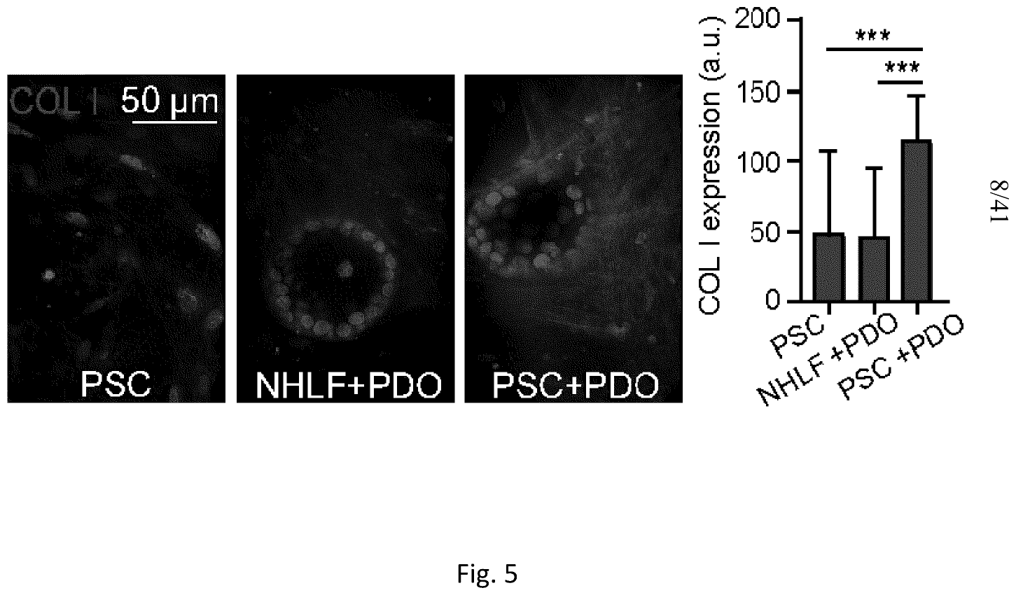

The excessive deposition of ECM, mainly induced by myCAFs, facilitates tumor growth and serves as a barrier impairing drug delivery and effector T-cell infiltration can be characterized on chip. For example, Collagen I, a major component in PDAC ECM, was found excessively deposited on chip, recapitulating the desmoplasia caused by myCAFs (FIG. 5). To study the components and density of ECM, key ECM components shaped by CAFs including collagens, integrins, and hyaluronic acid (HA) were stained. The evolution of CAFs subpopulations and ECM components/densities were characterized on day 1, 3, 5 and 7, and comparisons between chips with basal-like and classical subtypes of PDOs were conducted. The impact of dense ECM on molecule transport on chip was validated. Specifically, three conditions with different stromal niche: PSCs alone, NHLFs+PDOs and PSCs+PDOs were compared. After the stromal and tumoral niche is fully formed, Dextran Alexa Fluor™ 647 (Thermo Fisher) was infused into the outer ring of the chip, the diffusion of dextran into the tumoral niche was monitored in time-lapse manner, and the penetration rate of dextran was calculated. To validate the desmoplasia niche on chip recapitulating the in vivo ECM characteristics, the specific types and densities of the ECM components (collagens, integrins, HA etc.) on chip were compared with in vivo tumor tissue. The differences on percentage of major ECM components between on chip and in vivo niches should be within ±25%, and the density of ECM should increase >50% after PDOs were loaded on chip.

### Recapitulating the Hypovascularized Stromal Niche on PDAC Organoids-On-a-Chip

The desmoplastic stroma in TME causes the collapse and ablation of vasculature through the TGF-β receptor signaling pathway [Nguyen D-H T et al., A biomimetic pancreatic cancer on-chip reveals endothelial ablation via ALK7 signaling. Science Advances. 5(8):eaav6789] or the high IFPs generated by stromal barriers like HA [Provenzano P P et al., Enzymatic targeting of the stroma ablates physical barriers to treatment of pancreatic ductal adenocarcinoma. Cancer Cell. 2012; 21(3):418-29], modelling the unique inherently hypovascularized stromal niche of PDAC. Therefore, the formation of such poorly vascularized niche was validated by monitoring the change of the vascular network in 7 days after PDAC organoids were loaded on the chip with established perfusable and intact vasculature (FIG. 6A). The dynamic change of vasculature including the vessel area, diameter and height of the vasculature were measured daily under a confocal microscope (Nikon C2i) and analyzed by AngioTool (FIG. 6B). The intactness of the vessels were assessed by quantifying the junctions formed by HUVECs on vascular walls (staining CD31, FIG. 6C). Furthermore, the apoptosis of vasculature induced by PDOs was measured by Caspase 3/7 activity (Biotium, FIG. 6D). As shown in the results in FIG. 6, it was expected that after PDOs were grown on the chip, vasculatures would start to collapse, shrink, and finally transforms into hypovascularized status. PDAC patients with basal-like or classical subtypes were compared to find out whether subtypes effect the invasion of PDAC to vasculature and the stroma niche.

To validate the CAFs-induced hypovascularity, as organoids grow on the chip, interconnection between the tumor and stromal niche should be achieved, but the vasculature should collapse with the average height of vasculature <40 μm, the vascular network should be invaded with <50% area of the outer ring 7 days after organoids loading. It was expected that the number of junctions decreased to <40 per 10⁻¹ mm² (FIG. 6C), apoptosis of vascular cells induced by PDOs characterized by Caspase 3/7 activity were >30% (FIG. 6D), and the vasculature became leaky with the permeability >1.6×10⁻¹⁰ m²/s.

### In Vitro Examining the Subtype-Specific Immunosuppressive Characteristics in the PDAC Chip

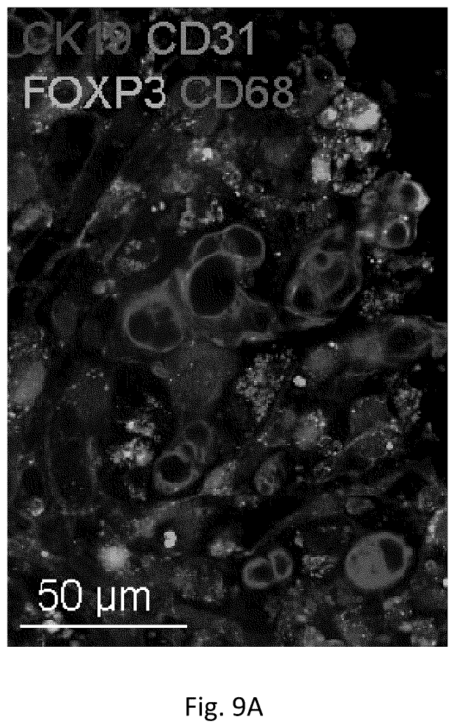

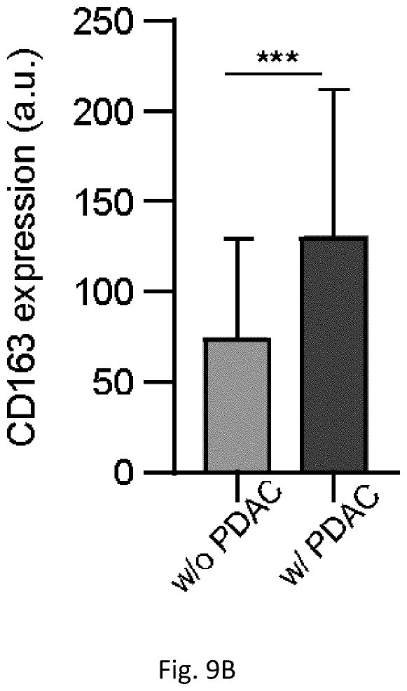

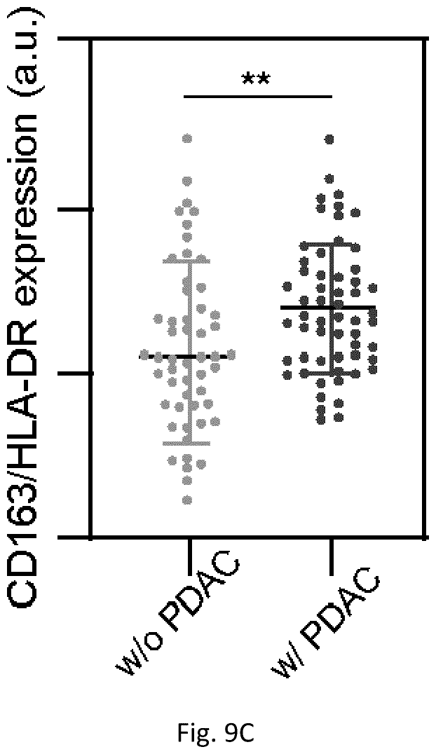

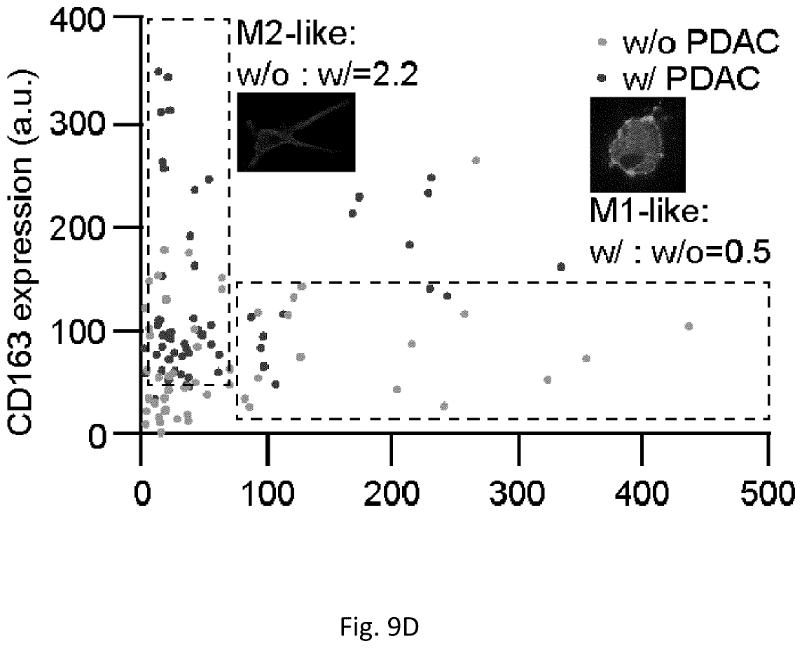

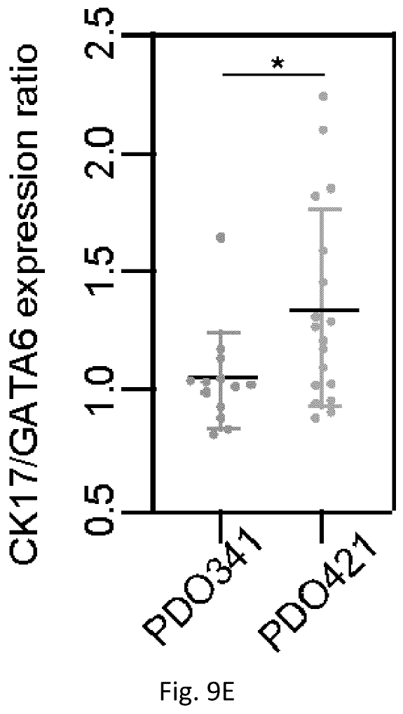

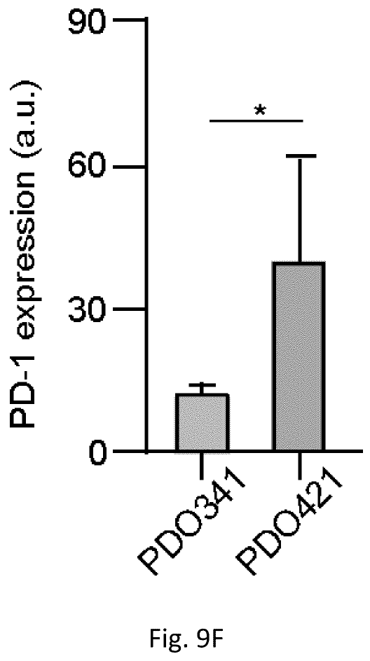

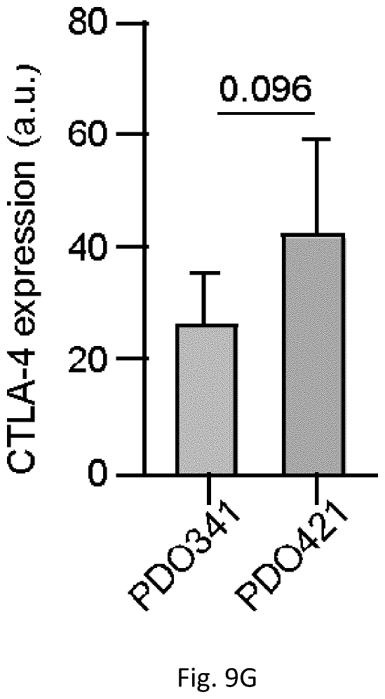

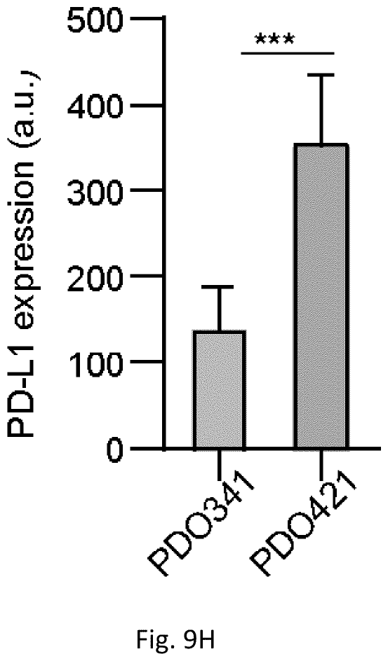

Key immune cell populations were loaded (TAMs, Treg cells, and MDSCs) and immune landscapes (e.g., immunophenotypes, immune checkpoint signaling, and cytokine profiles, FIG. 8) of different PDAC subtypes (6 basal-like and 6 classical) were characterized. Firstly, three experimental groups were set as classical and basal-like subtype based on RNA sequencing and biomarkers like CK17 (basal-like) and GATA6 (classical) (FIG. 9E) [O'Kane G M et al., GATA6 Expression Distinguishes Classical and Basal-like Subtypes in Advanced Pancreatic Cancer. Clinical Cancer Research. 2020; 26(18):4901-10; Roa-Peña L et al., Keratin 17 identifies the most lethal molecular subtype of pancreatic cancer. Sci Rep. 2019; 9(1):11239], and a negative control group (without PDOs), and investigated the interactions among TAMs, Treg cells, and MDSCs in the PDAC chip. TAMs, CD4⁺CD25⁺FOXP3⁺ Treg cells and CD11b⁺CD14⁻CD15⁺ MDSCs can be introduced on chip by mixing them with PDOs and seeding in a density mimicking the in vivo TME of original tumors (FIG. 9A, see Methodologies for details). The disclosed study showed that with the presence of PADC, TAMs were inclined to the CD163⁺ protumor-M2 phenotype with lower HLA-DR expression (FIGS. 9B through 9D). The phenotypes of infiltrating TAMs in basal-like or classical subtypes with M2-specific (CD163, CD206) and M1-specific (HLA-DR, CD80) markers were investigated [Zhou W et al., Periostin secreted by glioblastoma stem cells recruits M2 tumour-associated macrophages and promotes malignant growth. Nature cell biology. 2015; 17(2):170; Komohara Y, Jinushi M, Takeya M. Clinical significance of macrophage heterogeneity in human malignant tumors. Cancer science. 2014; 105(1):1-8].

Apart from TAMs, it was confirmed that Treg cells (FOXP3⁺) promote the immunosuppression by the upregulation of immune checkpoints like CTLA-4, PD-1, TIM-3 and LAG-3 [Goulart Et al., T cells in pancreatic cancer stroma. World J Gastroenterol. 2021; 27(46):7956-68; Gao Z et al., Advance of T regulatory cells in tumor microenvironment remodeling and immunotherapy in pancreatic cancer. European Journal of Inflammation. 2022; 20:1721727X221092900] using immunostaining and flow cytometry (FIGS. 9F & 9G). MDSCs in PDAC TME highly express CD80, can influence the differentiation of Treg cells through the CTLA-4/CD80 pathway [Fan J-q et al., advances and outlooks in immunotherapy for pancreatic ductal adenocarcinoma. Mol Cancer. 2020; 19(1):32], directly suppress T-cell activation through PD-1/PD-L1 pathway [Pergamo M, Miller G. Myeloid-derived suppressor cells and their role in pancreatic cancer. Cancer Gene Ther. 2017; 24 (3): 100-5]. Thus, PD-L1 and CD80 expression were measured on MDSCs in different PDAC subtypes.

Secondly, to study the secretion and dynamics of representative immunosuppressive cytokines in the TME, the culture medium from the chip was collected to measure cytokines by ELISA (Biolegend). Activated M2 TAMs can release immunosuppressive cytokines such as CCL2, IL-10, and TGF-β to promote intratumoral CD4⁺CD25⁺Foxp3⁺ Treg cell trafficking via CCL2/CCR4 [McGeachy M J et al., TGF-β and IL-6 drive the production of IL-17 and IL-10 by T cells and restrain TH-17 cell-mediated pathology. Nature immunology. 2007; 8(12):1390-7] and can release factors such as EGF and IL-6 to promote tumor cell growth and invasion using proliferation and cytotoxicity assays. The immunosuppressive role of Treg cells in PDAC TME was investigated by measuring key cytokines like TGF-β, IL-10 and IL-35 [Goulart et al., T cells in pancreatic cancer stroma. World J Gastroenterol. 2021; 27(46):7956-68; Gao et al., Advance of T regulatory cells in tumor microenvironment remodeling and immunotherapy in pancreatic cancer. European Journal of Inflammation. 2022; 20:1721727X221092900]. The overall cytokine profile was obtained by Human Cytokine Array (RayBiotech) as well.

Altogether, it was determined how molecularly different PDAC incur distinct immunosuppressive characteristics, resulting in their different prognoses and immunotherapy efficacy [Krieger T G et al., Single-cell analysis of patient-derived PDAC organoids reveals cell state heterogeneity and a conserved developmental hierarchy. Nature Communications. 2021; 12(1):5826; Miyabayashi K et al., Intraductal Transplantation Models of Human Pancreatic Ductal Adenocarcinoma Reveal Progressive Transition of Molecular Subtypes. Cancer Discov. 2020; 10(10):1566-89]. It was expected that the more basal-like subtype (e.g. PDO421 in FIG. 9E) demonstrates an “immune escape” mechanism with a more hostile TME richer in CD163⁺ M2-polarized TAMs and Treg cells with elevated immune checkpoints (PD-1, PD-L1, CTLA-4 see FIGS. 9F through 9H) and cytokines (TGFβ, IL-6, IL-10, IL-35, CCL2, and EGF) [Bailey P et al., Australian Pancreatic Cancer Genome I. Genomic analyses identify molecular subtypes of pancreatic cancer. Nature. 2016; 531(7592):47-52; Facciabene A, Motz G T, Coukos G. T-Regulatory Cells: Key Players in Tumor Immune Escape and Angiogenesis. Cancer Research. 2012; 72(9):2162-71]. However, the more classical subtype (e.g. PDO341 in FIG. 9E) is with an “immune rich” TME, characterized with more HLA-DR⁺ M1-polarized macrophages, and lower level of suppressive immune cells like Treg cells and M2-polarzied macrophages [Karamitopoulou E. Tumour microenvironment of pancreatic cancer: immune landscape is dictated by molecular and histopathological features. British Journal of Cancer. 2019; 121(1):5-14].

### How Stromal Niche Influences the Immunosuppressive PDAC TME—Exploring the CAF-Mediated Immune Response in PDAC Niche

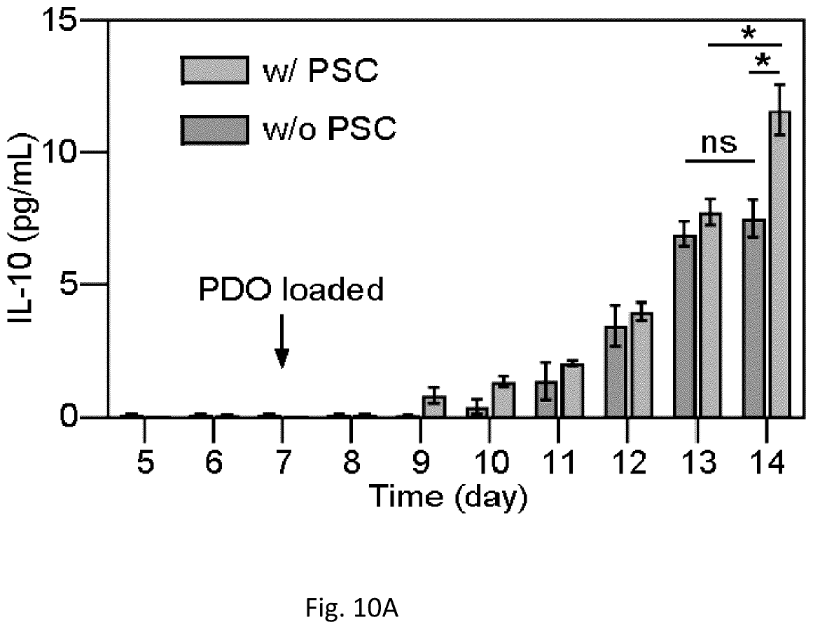

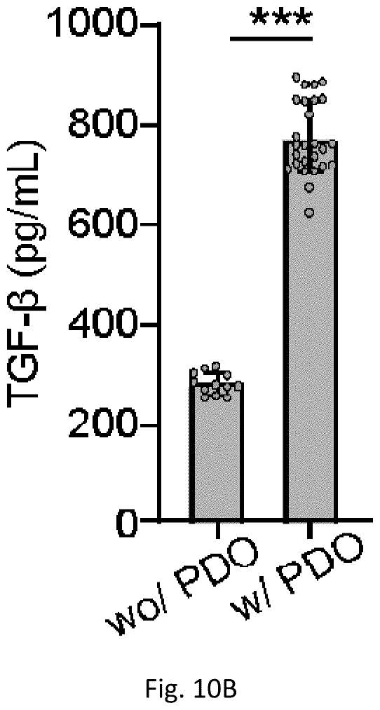

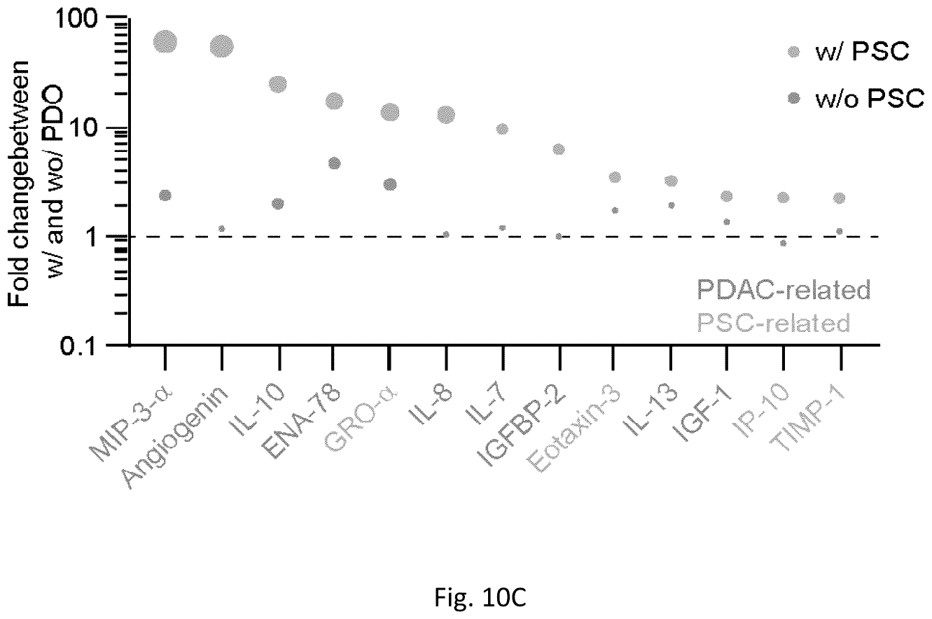

CAFs not only model a stromal niche of desmoplasia acting as a physical barrier for drug delivery and CTL infiltration, but also directly contribute to the immunosuppressive PDAC TME by secreting cytokines and chemokines, thus promoting tumor initiation, progression, invasion, and metastasis [Fearon D T. The Carcinoma-Associated Fibroblast Expressing Fibroblast Activation Protein and Escape from Immune Surveillance. Cancer Immunology Research. 2014; 2(3):187-93]. For example, the experiments found that anti-inflammatory cytokines like IL-10 and TGF-β were upregulated after PDOs were seeded on chip, in particular with higher secretion levels as PSCs existed comparing with the control group (FIGS. 10A & 10B). The cytokine array (RayBiotech) profiling indicated PSCs overall enhanced immunosuppression (FIG. 10C). To study the unique immune responses in PDAC niche modeled by iCAFs, firstly, ELISA was applied to record the dynamics of IL-6 (the main inflammatory cytokine in PDAC TME secreted by iCAF) and IL-1α (the cytokine inducing iCAF differentiation through IL1/JAK-STAT3 pathway secreted by cancer cells [Biffi et al., IL1-Induced JAK/STAT Signaling Is Antagonized by TGFβ to Shape CAF Heterogeneity in Pancreatic Ductal Adenocarcinoma. Cancer Discovery. 2019; 9(2):282-301]) with or without PDAC PDOs. Secondly, the CXCL12/CXCR4 pathway mediated by iCAFs led to decreased infiltration of CTL and resistance to chemotherapies [Öhlund D et al., Distinct populations of inflammatory fibroblasts and myofibroblasts in pancreatic cancer. Journal of Experimental Medicine. 2017; 214(3):579-96]. Thus, the secretion level of CXCL12 and expression level of CXCR4 were compared between chips with or without CAFs to investigate the protumor function of iCAFs.

### Example 2: A Pancreatic Cancer Organoids-On-a-Chip Model for CAR T-Cell Therapy Screening

Chimeric antigen receptor (CAR) T-cell therapy have been remarked as novel therapeutic strategies in pilot clinical trials for different tumors. However, the efficacy of CAR T-cell therapies in solid tumors, including pancreatic ductal adenocarcinoma (PDAC), is limited by a prohibitive tumor microenvironment (TME). The tumor immunosuppression and microenvironmental heterogeneity have emerged as potential barriers in efficient PDAC CAR T-cell treatment. However, the lack of an accurate methodology to quantitatively characterize the heterogeneous immune signatures of a patients' TMEs remains a critical unmet need in studying mechanisms of resistance and screening novel CAR T-cell therapy for PDAC. Discrepancies between the preclinical and clinical results have raised major concerns about the predictive value of current animal models for cancer immunotherapy testing, since they fail to reflect the tumor heterogeneity and human immunity—necessitating the pursuit of humanized models for assessing CAR T-cell therapies. Recent patient-derived tumor organoids (PDOs) can maintain molecular characteristics of the original tumor and allow for assessing new anticancer drugs, but they suffer from the drawback of not preserving the context of the patient's TME immune signatures and architectures (i.e., tumor vasculature, hypoxia, and physiological perfusion) that confer immunotherapeutic resistance. In order to develop more efficacious therapeutics, better model systems that can accurately recapitulate the immune response and pathological process of PDAC during CAR T-cell treatment are critically required. To address this unmet need in immune-oncology research, this disclosure provides a patient-derived “PDAC organoids-on-a-chip”microphysiological system by integrating the state-of-the-art organoids and organ-on-a-chip technologies to accurately reconstitute the PDAC TMEs in vitro for CAR T-cell therapy modeling and screening. The disclosed, synergistically engineered patient-derived ‘PDAC organoids-on-a-chip’ addresses each approach's limitations and synergistically combines the best features of each approach. The disclosed system better retains the inherent traits and the function of intact tumor tissues in order to assess, optimize and personalize CAR T-cell therapy strategies. Overall, the disclosed system demonstrates a new paradigm for “clinical trials on a chip” studies that can improve the efficacy of CAR-T cell therapy in solid tumors.

### Innovative Cancer Technology: PDAC Organoids-On-a-Chip for CAR T-Cell Therapy Screening

Organ-on-a-chip and PDOs represent two fundamentally different yet complementary approaches toward the same goal of recapitulating the complexity of human organs in vitro [Park S E, Georgescu A, Huh D. Organoids-on-a-chip. Science. 2019; 364(6444):960-5]. Yet, Organ-on-a-chip or PDOs alone have limited capacity to meet the broad range of needs that arise in PDAC CAR T-cell therapy and TME modeling. It was hypothesized that a strategic integration of the two emerging technologies, i.e., integrating PDAC PDOs on a vascularized TME chip with biomimetic stromal and immune niches, may address each approach's limitations and synergistically combine the best features of each approach to provide a more powerful in vitro technology for PDAC CAR T-cell therapy modeling and screening [Takebe T, Zhang B, Radisic M. Synergistic Engineering: Organoids Meet Organs-on-a-Chip. Cell Stem Cell. 2017; 21(3):297-300; Park S E, Georgescu A, Huh D. Organoids-on-a-chip. Science. 2019; 364(6444):960-5; Zhang S, Wan Z, Kamm R D. Vascularized organoids on a chip: strategies for engineering organoids with functional vasculature. Lab Chip. 2021; 21(3):473-88)].

Thus, the objective of this disclosure was to develop a patient-derived “PDAC Organoids-on-a-Chip” micro-physiological system by strategic integration of the state-of-the-art organoids and organ-on-a-chip technologies to accurately reconstitute the pathophysiology and heterogeneity of the PDAC TME in vitro, in order to study mechanisms of resistance, assess, optimize, and personalize CAR T-cell therapies (FIG. 2). The proposed in vitro chip model retains the cellular heterogeneity and phenotype fidelity of PDAC PDOs, and recapitulates the stromal niche of PDAC TME with biological and biophysical control functions. Immune niche of PDAC TME was also dissected on chip to shed light on the mechanisms of how immune cells interact and together shape the subtype-specific immunosuppressive TME of PDAC. By characterizing the functions of CAR T-cells, dissecting the immunosuppressive TME, and mapping the therapy response, the PDAC Organoids-on-a-Chip can serve as a preclinical model to rapidly test, assess and screen candidate CAR T-cell products of different designs.

### Evaluating CAR T-Cell Therapy on the PDAC Organoids Chip

The heterogenous expressions of tumor-specific antigens on PDAC and the inherently immunosuppressive TME pose challenges on the development of efficacious CAR T-cell therapies. The patient-derived PDAC organoids-on-a-chip system that recapitulates the heterogenous immunosuppressive TMEs permits a multidimensional examination of CAR T-cell therapy at high spatiotemporal resolution, subsequently screening of effective counterstrategies. To mechanistically understand how CAR T-cell function in patient tumor niche and screen candidate effective tumor-specific antigens/costimulatory domains of CAR T-cells towards patient-specific PDAC TME, a protocol to evaluate tumor responses to CAR T-cell therapy was established.

### Engineering a Patient-Derived In Vitro PDAC Organoids-On-a-Chip Model to Reconstitute the In Vivo Stromal Niche of the PDAC TME

First, a tissue engineered human PDAC microphysiological system was developed with PDOs and key stromal niche components including vasculature and cancer-associated fibroblasts (CAFs) to replicate their in vivo counterparts. Second, it was confirmed that main in vivo stromal niche features like the upregulation of CAF-related markers, hypovascularity and dense ECM were retained on chip. The disclosed organoids-on-a-chip model established the in vivo desmoplasia of PDAC stromal niche.

### Reconstituting and Dissecting Immunosuppressive Characteristics of PDAC TME on Chip

First, the immune niche on chip was reconstituted by combining main immune components like tumor associated macrophages (TAMs), regulatory T cells (Treg cells) and myeloid-derived suppressor cells (MDSCs). The cellular (immune cell infiltrate composition, phenotypes, and dynamics), genomic and epigenetic (DNA), transcriptomic (RNA), and proteomic (cytokines) signatures of the PDAC TME were mapped. Second, stromal niche factors were studied, such as how CAFs mediate immunosuppressive responses by examining cytokine profiling and key signaling pathways. Thus, the disclosed organotypic tumor model reconstituted significant hallmarks of the in vivo immune niche and enabled a multiparametric analysis of the pathologically-relevant TME with increased specificity and sensitivity.

### Modeling CAR T-Cell Therapy on the PDAC Chip

A standard CAR T-cell product (Mesothelin scFv-4-1BB-CD3ζ, ProMab) [Akce M et al., The Potential of CAR T Cell Therapy in Pancreatic Cancer. Front Immunol. 2018; 9:2166; DeSelm C J, Tano Z E, Varghese A M, Adusumilli P S. CAR T-cell therapy for pancreatic cancer. Journal of Surgical Oncology. 2017; 116(1):63-74] was adopted targeting Mesothelin (MSLN), a highly expressed marker in PDAC, to model CAR T-cell therapy on chip. Anti-MSLN CAR T-cells were infused on the chip through the 3D perfusable microvessel in various densities (e.g., 2-5×10⁶ cells/ml) and ratios (e.g., CAR T-cell:tumor=1:10-1:1) and characterize their functions and immune responses. No transduced (MOCK) T-cells isolated from PBMCs were loaded on chip under the same condition as control.

To monitor CAR T-cell activation and proliferation, the expressions of T-cell activation markers were characterized, such as CD25, CD154, CD69, interferon gamma, granzyme B and perforin, and proliferation markers (Ki-67) using immunostaining and PCR. It was expected to observe a strong activation and increased proliferation of CAR T-cells on chip comparing with that of MOCK T-cells within 3 to 6 days (as indicated in FIGS. 11A and 11B).

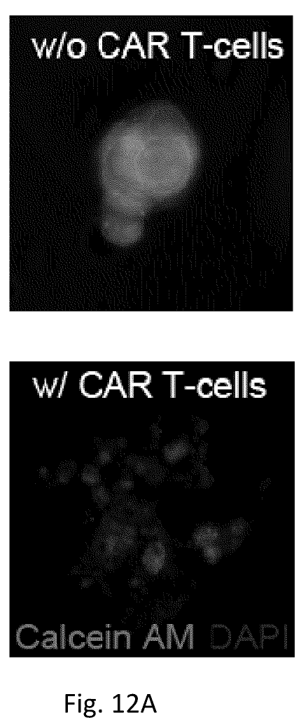

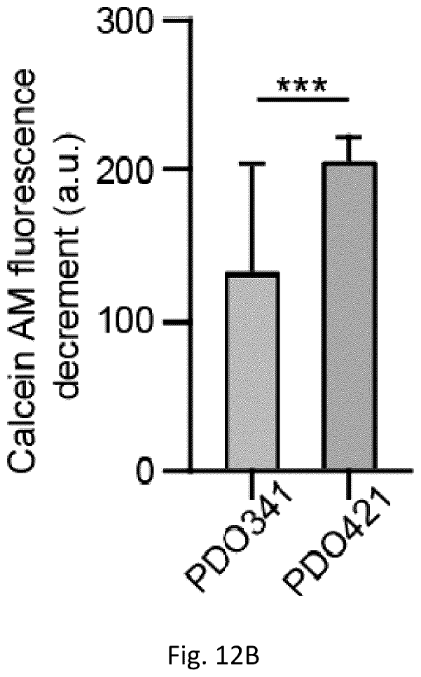

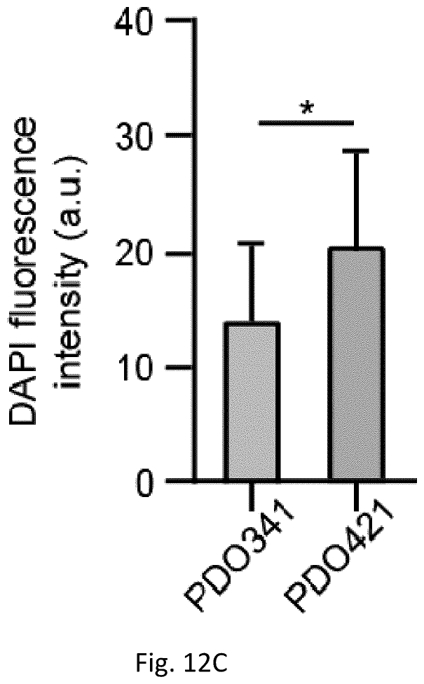

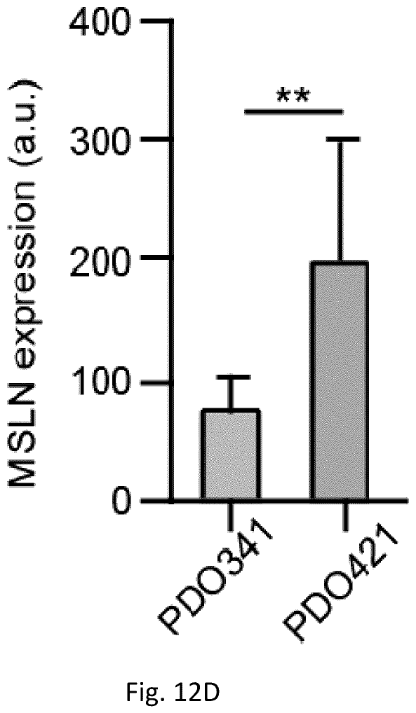

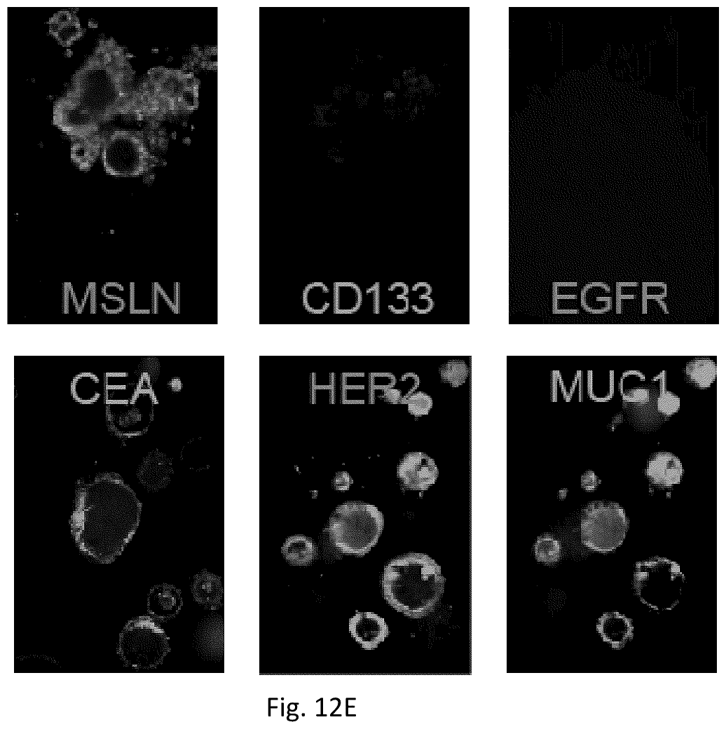

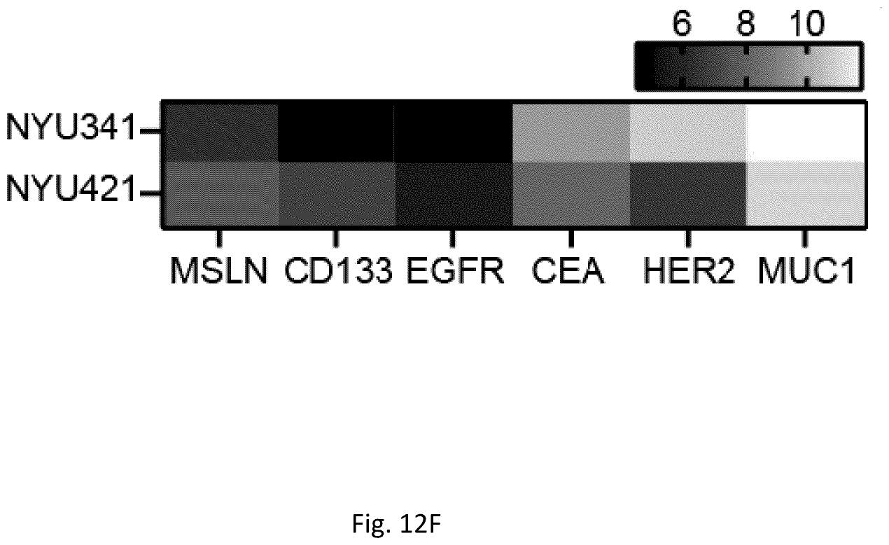

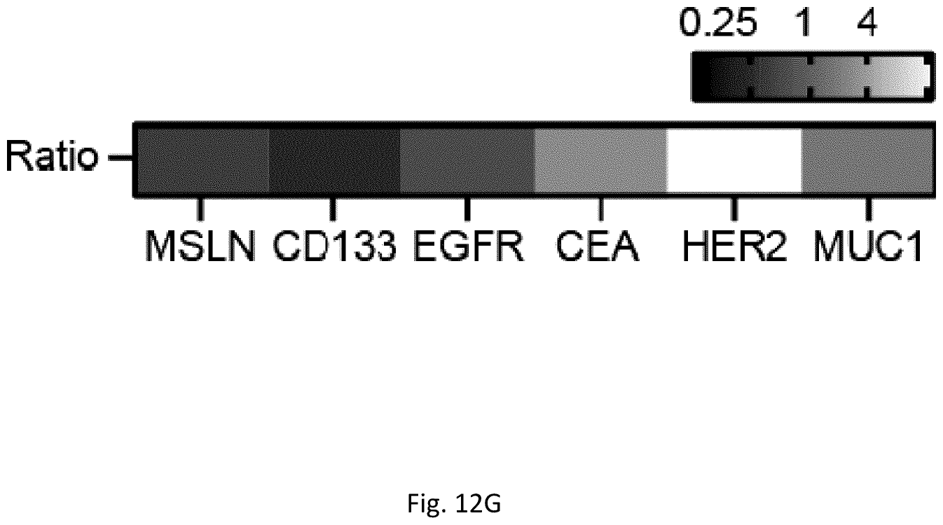

To study the CAR T-cell cytotoxic activity, the tumor apoptosis rate was longitudinally monitored (Nuc View® 405 Caspase-3 Substrate, Biotium) [Cui X et al., Dissecting the immunosuppressive tumor microenvironments in Glioblastoma-on-a-Chip for optimized PD-1 immunotherapy. Elife. 2020; 9:e52253] over 1 week in the device to find if the tested different patients could achieve on-chip remission, stable disease, or progression to CAR T-cell therapy. The data shows the patient-specific cytotoxicity by live/dead cells staining (FIGS. 12A through 12D). The effect of CAR T-cell dose (e.g., CAR T-cell:tumor=1:10 to 1:1) on CAR T-cell activation, proliferation and the clearance time of PDAC cells was also tested.

To assess the immune response in TME, conditioned media was collected daily from the chip, and key cytokines such as TNF-α and IFN-γ were measured by ELISA and compared among conditions. The study results already showed that CAR T-cell induced the highest level of inflammatory responses relative to the control groups with MOCK T-cells or without T-cells (FIG. 11F). Overall, responses of CAR T-cells in different subtypes of PDAC TME were compared to confirm if subtypes lead to distinct functional responses.

### Monitoring the CAR T-Cell Trafficking Dynamics in the Tumor Niche

Due to the PDAC TME with dense stroma, hypovascularity, abundant immunosuppressive cells and cytokines, the infiltration of CAR T-cells might be hindered. Therefore, the infiltration and migration of CAR T-cells on chip were monitored to confirm the effects of the tumor immune and stromal niche (densities of ECM, amounts of CAFs, and levels of hypovascularity) on CAR T-cells trafficking. To clearly distinguish the cell infiltrations during live imaging, CAR T-cells and PDAC organoids were pre-labelled with dyes such as CellTracker Red and DiD (Thermo Fisher). The dynamic extravasation from vessel and migratory patterns of CAR T-cells into the PDAC TME were longitudinally studied on a daily basis for 7 days with live cell imaging under confocal microscopy (as shown in FIG. 11C and [Cui X et al., Dissecting the immunosuppressive tumor microenvironments in Glioblastoma-on-a-Chip for optimized PD-1 immunotherapy. Elife. 2020; 9:e52253]). The CAR T-cell extravasation rate, migration trajectories, distance and speed were quantified in different patient-derived TMEs with ImageJ (NIH) (FIG. 11E). Together, the disclosed Chip model reveals that both the immune and stromal niche influence the CAR T-cell trafficking, and CAR T-cells exhibit distinct kinetics in different subtypes of PDAC related to treatment response, which cannot be easily examined in vivo due to technical limitations.

CAR T-cells were pre-labelled with fluorescent dye and the trajectory was monitored and recorded. To achieve an ideal CAR T-cell infiltration for cytotoxicity, it was expected that >30% of the CAR T-cells should traffic into the tumor niche in 2 days; in 4 days, >50% of CAR T-cells should be distributed within 200 μm distance from PDOs. The infiltrations of CAR T-cells in niches with or without PDOs were recorded for comparison to confirm that CAR T-cell infiltration is affected by PDAC-reshaped stromal and immune niches.

### Examine how PDAC Immune Niche Influences CAR T-Cell Therapy

To understand the resistance mechanisms contributed by PDAC immune niche, the evolution of PDAC immune niche treated with CAR T-cell therapy were longitudinally monitored, and how different subtype- and patient-specific TME immune landscapes was studied (e.g., immune cell populations, their immunophenotypes, and cytokine profiles measured regulate PDAC response to CAR T-cell therapy). It was tested whether niche immune cells, in particular, TAMs, Treg cells and MDSCs can induce immune escape by inhibiting CAR T-cell activation, proliferation, and cytotoxicity. It was expected that upregulated PD-L1 expression on TAMs and MDSCs arrested the CD154⁺ activation of CAR T-cell either physically or immunologically in PDAC niche. Whether TAMs delay CAR T-cell infiltration and killing dynamics were confirmed by measuring CAR T-cell migration trajectories and tumor apoptosis rate. ELISA was deployed for a longitudinal multiplex monitoring of the dynamic immunological secretomic signatures (e.g. IL-10, TGF-β, IL-6 and CCL2, as shown in FIG. 10) of tumor immunity that signify treatment outcomes of remission, stable disease or resistance over a week. scRNA-Seq tool was used to comparatively analyze the cellular population, cytokine pathways, and gene expression in CAR T-cell and PDAC niche cells among patients of basal-like and classical subtypes (as discussed in [Ma C et al., Leukemia-on-a-chip: Dissecting the chemoresistance mechanisms in B cell acute lymphoblastic leukemia bone marrow niche. Sci Adv. 2020; 6(44)]) to identify determinants in regulating remission, resistance, or relapse post CAR T-cell therapy. Altogether, it was confirmed that the patient-specific immunosuppressive milieu differentially regulates TAMs, Treg cells and MDSCs, hinders the CAR T-cell infiltration, activation, and cytotoxic function in different PDAC subtypes.

### Screening Different CAR T-Cell Therapies on Chip

The heterogeneity of PDAC tumor cells result in various expression of surface antigens, creating a challenge to select bona-fide pancreatic cancer-specific antigens targeted by CAR T-cells achieving high cytotoxicity and low adverse events [Akce M et al., The Potential of CAR T Cell Therapy in Pancreatic Cancer. Frontiers in Immunology. 2018; 9; Yeo D, Giardina C, Saxena P, Rasko J E J. The next wave of cellular immunotherapies in pancreatic cancer. Molecular Therapy-Oncolytics. 2022; 24:561-76]. Potential antigens like MSLN, HER2 (expressed on 20-60% PDACs), CD133 (highly expressed in PDAC cancer stem cells), CEA (expressed on nearly 75% PDACs), MUC1 (expressed on 90% PDACs with predictable O-linked glycosylation) and PSCA (expressed on 60-80% PDACs with low basal expression on normal tissues) have been tested in clinical trials [DeSelm C J et al., CAR T-cell therapy for pancreatic cancer. Journal of Surgical Oncology. 2017; 116(1):63-74]. The expression levels of these main PDAC surface antigens (MSLN, HER2, EGFR, CD133, CEA, MUC1, and PSCA) on different PDAC PDOs were first profiled by immunostaining or flow cytometry (as shown in FIGS. 12E through 12G). CAR T-cells (ProMab Biotech) targeting MSLN, HER2, CD133, EGFR and CEA, respectively, were then applied and the efficacy each type of CAR T-cells in different patients on the PDAC chip was characterized. It was confirmed that the CAR T-cell efficacy is highly relevant to the expression level of specific surface antigen on each line of PDOs.

Secondly, apart from antigen selection, the costimulatory domain of CAR T-cells can affect responses as well: CD28 domain induced rapid expansion of CAR T-cells while 4-1BB domain increased the persistence of CAR T-cells with low expansion rate. CAR T-cell products with different costimulatory domains were applied (e.g., anti-MSLN with CD28 or 4-1BB domain; ProMab Biotechnologies) on chip and compared with treatment outcomes.

### Indices for Multidimensional Analyses of CAR T-Cell Function, TME Immunosuppression, Therapeutic Responses

To systematically and quantitively assess CAR T-cell therapy screening results from different dimensions, 3 key indices normalized with range of 0-1 were used to evaluate CAR T-cell functions, TME immunosuppression and therapeutic responses. Firstly, experimental measurement quantities were normalized for nondimensionalization and further comparison. CAR T-cell function index: CAR T-cell infiltrate frequency, speed, and distant, expression levels of activation (CD154, CD69), proliferation (Ki67) and cytotoxic function (interferon gamma, granzyme B and perforin) markers.

Immunosuppression index: relative frequencies and immunosuppressive function of TAM (CD163, Fizz-1, Arg1, CSF-1) and Treg cell (CD4+CD25+Foxp3+, 5 CTLA-4), immunosuppressive cytokine profile (e.g., TGF-β, IL-10, CCL2) and expression level of immune checkpoints markers (e.g. PD1/PDL1) were evaluated. Response index: the expression level of targeted antigens on tumor and the apoptosis rate of tumor cells were evaluated. The responses of PDOs on chip were classified into three categories: remission, stable disease and progression. Remission was defined as the number and average diameter of the PDOs decrease by 50% (or apoptosis rate larger than 50%), progression was defined as the number and average diameter of the PDOs increase by 25%, and stable disease was defined as neither remission nor progression.

### New Capacities and Transformative Potential

CAR T-cell therapy screening: This study demonstrated a unique precision immuno-oncology platform that recapitulated the patient-specific tumor pathophysiology and host immunity that allows for a rapid (2-3 weeks) and accurate assessment of novel CAR designs (antigens, costimulatory domains) for PDAC treatment.

Monitoring CAR T-cell trafficking at high spatiotemporal resolution: The disclosed platform enables a real-time monitoring of CAR T-cells trafficking dynamics to a solid tumor and involves numerous sequential steps that include transport in the vessel, extravasation into the TME, migration, recognition of the tumor cells, and tumor cell killing.

PDAC TME modeling: The tissue engineered patient-derived PDAC Organoids-on-a-Chip microphysiological system can retain the cellular heterogeneity and phenotype fidelity of original tumor and reduce inter-organoid variability. The PDAC chip model allows one to investigate the immunomodulatory mechanisms in the pancreatic niche underlying PDAC resistance to CAR T-cell therapy, and screen new TME-associated biomarkers and treatment regimens to improve patient response to CAR T-cell therapy.

Personalized immunotherapy development: Such a patient-derived cancer chip system can be incorporated into clinical trials and provide a new paradigm for “clinical trials on a chip” studies that can help screen new biomarkers and potential responders, and significantly accelerate the pace for developing personalized immunotherapeutic strategies for PDAC patients to improve clinical responses.

Results provided direct evidence that the disclosed patient-derived ‘PDAC organoids-on-a-Chip’ model is an effective precision medicine system to rapidly evaluate CAR T-cell therapies with different designs (antigens, costimulatory). While previously considered an immune privileged site with a very low level of T-cell infiltration, evidence also demonstrates that in some cases T-cell infiltrate and preferentially accumulate in PDAC [DeSelm C J et al., CAR T-cell therapy for pancreatic cancer. Journal of Surgical Oncology. 2017; 116(1):63-74]. The disclosed chip model thus revealed the kinetics of CAR T-cells towards patient-specific PDAC niche, which cannot be easily examined in current in vivo method.

To cross-validate the screening results on chip, the study included in vivo transplantation of PDOs into mice forming PDO xenograft (PDOX) models and in order to compare the responses with on-chip results. Greater than 75% responses of the on-chip and PDOX model were the same, therefore the PDAC organoids-on-a-chip platform achieved the goal as a preclinical screening tool. Moreover, if proposed antigens like MSLN, HER2, CD133, CEA, MUC1 and PSCA do not live up to expectations, the disclosed system may be implemented to screen other new CAR T cell designs in the system.

It was expected to obtain all the readouts from CAR T-cell therapy testing within 3 weeks, which is 4-8 times faster than the “gold standard” animal models (3-6 months) [Ariston Gabriel A N, Jiao Q, Yvette U, Yang X, Al-Ameri S A, Du L, Wang Y-s, Wang C. Differences between KC and KPC pancreatic ductal adenocarcinoma mice models, in terms of their modeling biology and their clinical relevance. Pancreatology. 2020; 20(1):79-88].

The following publications are incorporated by reference in their entirety:
Haque M R, et al. Patient-derived pancreatic cancer-on-a-chip recapitulates the tumor microenvironment. Microsystems & Nanoengineering. 2022; 8(1):36. doi: 10.1038/s41378-022-00370-6.
Homan K A, et al. Flow-enhanced vascularization and maturation of kidney organoids in vitro. Nat Methods. 2019; 16(3):255-62. Epub 2019 Feb. 12. doi: 10.1038/s41592-019-0325-y. PubMed PMID: 30742039; PMCID: PMC6488032.
Rajasekar S, et al. IFlowPlate—A Customized 384-Well Plate for the Culture of Perfusable Vascularized Colon Organoids. Advanced Materials. 2020; 32(46):2002974. doi: https://doi.org/10.1002/adma.202002974.
Tiriac H, et al. Organoid Profiling Identifies Common Responders to Chemotherapy in Pancreatic Cancer. Cancer Discovery. 2018; 8(9):1112-29. doi: 10.1158/2159-8290. CD-18-0349.
Ma C, et al. Leukemia-on-a-chip: Dissecting the chemoresistance mechanisms in B cell acute lymphoblastic leukemia bone marrow niche. Science Advances. 6(44):eaba5536. doi: 10.1126/sciadv.aba5536.
Hu Z, et al. Vascularized Tumor Spheroid-on-a-Chip Model Verifies Synergistic Vasoprotective and Chemotherapeutic Effects. ACS Biomaterials Science & Engineering. 2022; 8(3):1215-25. doi: 10.1021/acsbiomaterials.1c01099.
Cui X, et al. Dissecting the immunosuppressive tumor microenvironments in Glioblastoma-on-a-Chip for optimized PD-1 immunotherapy. eLife. 2020; 9: e52253. doi: 10.7554/eLife.52253.
Integrated human organ-on-chip microphysiological systems. Patent No.: U.S. Pat. No. 10,954,482 B2
Integrated organ-on-chip systems and applications of the same. Pub. No.: US 2019/0064144 A1
Organ chips and uses thereof. Pub. No.: US 2019/0032021 A1
Devices, systems and methods for inhibiting invasion and metastases of cancer. Pub. No.: US 2017/0285003 A1
Organ on chip integration and applications of the same. Patent No.: U.S. Pat. No. 10,119,622 B2
Identifying drug candidates for treating polycystic kidney disease using organoids generated from primary cells. Patent No.: U.S. Pat. No. 11,035,852 B2
Systems and methods for growth of intestinal cells in microfluidic devices Patent No.: U.S. Pat. No. 11,326,149 B2
Spontaneously beating cardiac organoid constructs and integrated body-on-chip apparatus containing the same. Pub. No.: US 2018/0273904 A1
Cell matrix optimization for formation of vascularized renal organoids, and development of organoid chip. Patent No.: WO2023282580A1

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A pancreatic cancer organoids on a chip device, comprising:
a microfluidic chip comprising a top and bottom surface;
a central chamber embedded in the microfluidic chip;
at least one opening in the top surface of the chip fluidly connected to the central chamber with one or more channels; and
a plurality of evenly spaced micropillars arranged in a substantially circular shape within the central chamber such that the central chamber is partitioned into at least a first inner region and a first outer region;
wherein the first inner region comprises an immunocompetent pancreatic region configured to mimic a pancreatic niche, and the first outer region comprises a vascular region configured to mimic an adjacent vascular network.

2. The device of claim 1, wherein the immunocompetent pancreatic region comprises a 3D pancreatic tumor niche.

3. The device of claim 1, wherein the first outer region further comprises a vascular region configured to serve as a pathway for nutrients, drugs and cells transport.

4. The device of claim 1, wherein the first inner region comprises one or more components selected from the group consisting of: hydrogel, pancreatic ductal adenocarcinoma (PDAC) cells, patient-derived organoids (PDOs), primary human umbilical vein endothelial cells (HUVECs), green fluorescent protein (GFP) expressing human umbilical vein endothelial cells (GFP-HUVECs), vasculature, extracellular matrix (ECM), culture media, cytokines, cancer-associated fibroblasts (CAFs), pancreatic stellate cells (PSCs), chimeric antigen receptor (CAR) T-cells, niche immune cells (tumor associated macrophages (TAMs), Treg cells, and myeloid-derived suppressor cells (MDSCs)).

5. The device of claim 1, wherein the first outer region comprises components selected from the group consisting of: primary human umbilical vein endothelial cells (HUVECs), green fluorescent protein (GFP) expressing human umbilical vein endothelial cells (GFP-HUVECs), vascular niche supporting cells: normal human lung fibroblasts (NHLFs), hydrogel, endothelial cells growth medium (EGM-2, Lonza), and Vascular endothelial growth factor (VEGF).

6. The device of claim 1, wherein the device is configured to replicate or mimic a pancreatic disease or disorder state selected from the group consisting of: pancreatic disease, pancreatitis, pancreatic cancer, and pancreatic ductal adenocarcinoma (PDAC).

7. The device of claim 6, wherein the device comprises CAR T-cells in the first outer region.

8. A method of determining pancreatic disease treatment responsiveness, comprising the steps of:
providing the device of claim 6;
administering a pancreatic disease treatment to the central chamber; and
determining pancreatic disease treatment responsiveness based on a measured change in the central chamber.

9. The method of claim 8, wherein the pancreatic disease treatment is a therapy selected from the group consisting of: anti-cancer drug, immunotherapy, chemotherapy, radiation therapy, chemoradiation therapy, and targeted therapy.

10. The method of claim 8, wherein the measured change comprises measuring a change in one or more parameters selected from the group consisting of: CAR T-cell infiltration frequency, speed, trajectory, and distance, expression levels of activation (CD25, CD154, CD69), proliferation (Ki67), cytotoxic function (interferon gamma, granzyme B, and perforin) markers, relative frequencies and immunosuppressive function of TAM (CD163, Fizz-1, Arg1, CSF-1) and Treg cell (CD4⁺CD25⁺Foxp3⁺, CTLA-4), immunosuppressive cytokine profile (e.g., TGF-β, IL-10, CCL2), expression level of immune checkpoints markers (e.g. PD1/PDL1), the expression level of targeted antigens on tumor and the apoptosis rate of tumor cells.

11. The device of claim 1, wherein the first inner region and the first outer region are concentric.

12. The device of claim 1, wherein the plurality of micropillars are evenly spaced by a distance between about 50 μm and 200 μm and have a cross-sectional shape selected from the group consisting of: circular, ovoid, square, rectangular, triangular, trapezoidal, and polygonal.
